(19) United States
(12) Patent Application Publication
(10) Pub. No.: US 2022/0157417 A1
(43) Pub. Date: May 19, 2022

(54) SYSTEM, METHOD AND USER INTERFACE FOR RECORDED INFORMATION

(71) Applicant: SUPPORTED PATIENT, LLC, Beverly Hills, CA (US)

(72) Inventor: Marci Reiss, Los Angeles, CA (US)

(21) Appl. No.: 17/438,376

(22) PCT Filed: Mar. 11, 2020
(86) PCT No.: PCT/US2020/022203

§ 371 Date: Sep. 10, 2021

Related U.S. Application Data
(60) Provisional application No. 62/816,218, filed on Mar. 11, 2019.
(60) Provisional application No. 62/849,081, filed on May 16, 2019.

(51) Int. Cl. G16H 10/60

(52) U.S. Cl. CPC G16H 10/60

(57) ABSTRACT

Disclosed herein are computer-implemented graphical user interfaces, systems, apparatuses and methods for accessing entity encounter information, such as electronic health record (EHR) information for a patient. Disclosed methods include displaying on a screen Patient Encounter Cards (PEC's) for a patient, where each PEC is card included useful at-a-glance information corresponding to the full entries in the EHR system related to a Patient Encounter (PE). Each PEC includes content extracted from its corresponding full entry. The novel GUI displays the PEC's on a vertical scrollable timeline in either forward or reverse-chronological order, thereby providing authorized viewers with easy and rapid access to relevant encounter information.

### RELATED APPLICATIONS

This application claims the benefit of U.S. Provisional Application No. 62/816,218 filed Mar. 11, 2019 and U.S. Provisional Application No. 62/849,081 filed May 16, 2019, the contents of each of which are incorporated by this reference in their entireties for all purposes as if fully set forth herein.

### TECHNICAL FIELD

The present invention relates generally to the field of electronic recordkeeping, and in particular, to systems, methods and graphical user interfaces for displaying and managing information from electronic records of or related to entities or individuals, such as clients, customers or patients.

### BACKGROUND

Systems and methods for collecting, storing, managing, searching for and presenting information in and from electronically-stored records are well-known. When electronic record systems (ERSs) are used to capture information relating to “entities” whether they be individuals, such as clients or patients, groups of individuals, or other individually-identifiable structures, such as things, companies or associations, this information is commonly stored in individually identifiable Entity Records (ERs) in the ERS. And, where the information captured results from “encounters”—events that (often but necessarily regularly) occur in time—it is often important that each “entity encounter” (EE) be reliably captured in an accurate, time-stamped, Entity Encounter Information Entry (EEIE) to be stored in the entity's ER of such ERS. An EEIE can be an entry entered by a human, it may be automatically populated by other input systems, or a combination of both. In large systems, many ER's may be regularly and simultaneously updated with new EEIEs related to EEs. It is thus understood that such larger ERS can rapidly grow to be huge in size with mounds of data collected and stored in short periods of time

Of course, in order for all this stored information to be useful to those people or systems that have a need to access and review it or parts of it, there is a need to efficiently identify and present electronically-stored EE information requested by authorized users on digital screens (such as computer displays or mobile devices) that are networked to the ERSs in ways that are useful to the users. Providing users with either too little summary information would be insufficient, and providing too much information to users, such as the entire EEIE on their screens, is overkill and cumbersome to manage.

Thus, what is needed is a system, method and user interface that intelligently selects requested information regarding an entity that is captured in time and stored in an ERS and presents relevant EE information to a user's digital screen in a user interface that allows for efficient review of such information.

What is needed is an EE visual interface, or graphical user interface (GUI), that overcomes the drawbacks of state of the art, widely used record retrieval and review systems.

Also needed is a system that seamlessly aggregates and presents content or data in a manner that allows for the quick visual retrieval of chronological EE pertinent to a particular subject or category.

Turning to the field of electronic health records, or EHR's, (sometimes also referred to as electronic medical records or EMR's), many commercial systems on the market exist to serve this purpose of managing patient information within a particular healthcare provider setting, such as the well-known EPIC™ and CERNER™ systems. EHR systems are essentially electronic versions of paper-based patient medical histories, where patient records are stored in customized, searchable databases (database management systems, or “DBMS”) used and often maintained by healthcare providers (hereinafter “Providers”) such as hospitals and medical practices. An EHR system Patient Record (“PR”) typically includes key administrative clinical data relevant to that person's care under a particular Provider, including basic patient demographic information, progress notes, presenting problems, medications, vital signs, past medical history, immunizations, laboratory data, radiology reports, etc. These EHR systems enable access to PR's using computing devices with displays (e.g. computers in examination or procedure rooms, or wireless mobile devices, etc.) that are networked or can connect to the EHR DBMS in order to search for and view PR's of interest and to input new patient data. Indeed, these systems have streamlined, and have the potential to significantly further streamline, the clinician's workflow. The EHR model also has the ability to support other care-related activities directly or indirectly through various interfaces, including evidence-based decision support, quality management, and outcomes reporting.

Moreover, EHR systems have long held out the promise of improving patient care and even transforming health care in a number of ways. To start, EHR's can reduce the incidence of medical error by improving the accuracy and clarity of medical records. EHR's can make health information readily accessible to any authorized user who needs such access. These systems hold the potential to make sharing patient information with patients and their guardians and other clinicians much easier and can reduce duplication of tests, reduce delays in treatment, etc. Significantly, they can also enable patients to be better and well-informed and thus make better decisions about their own care. Additional benefits of the modern EHR system can be found in numerous places online such as at https://www.healthit.gov/faq/what-are-advantages-electronic-health-records.

The conventional EHR system of the type used and often customized by a single Provider, such as a hospital system, generally operates as follows. Initially, a Patient Record (PR) for a patient is created in the Provider's EHR containing basic patient information (e.g., name, age, contact information, etc.), the date the PR was created, and a unique Patient ID number that the system assigns to that patient. The PR is stored in a HIPAA secure database environment. The Provider's authorized personnel or systems, such as doctors, physician assistants, nurses, as well as in-house or affiliated labs, imaging centers and other patient data collecting systems, can then enter information for each encounter with the patient, or Patient Encounter (PE), into the patient's PR. We will call each such entry, whether entered manually or automatically, a Patient Encounter Information Entry (PEIE). One type of PEIE that will be entered (often, most the prevalent type) is notes summarizing visits with or phone calls to the patient's doctors, PA's, nurses or other medical or mental healthcare providers. These notes, often called Progress Notes (PN's) or other names in various EHR systems, hereinafter will be referred to as PN's to denote text entries (and/or other formats) entered in a patient's PR related to an encounter with that patient. PEIE's may also comprise many other information types, including but not limited to data or reports related actions taken by, to or from a patient, such as diagnostic procedures (e.g., results of blood or urine tests performed at a lab, images and assessment reports from imaging procedures, or pathology reports on biopsies, etc.) and therapeutic procedures (e.g., surgeries), etc. It will be understood that PEIE's may be entered into a Patient Record in any myriad of ways known today or later developed. Means for creating PEIE's in a PR may include entering PN's in electronic Progress Note input forms displayed on networked devices (e.g., computers, tablets, and mobile phones) that are logged into the EHR system by the authorized personnel, uploading to the PR PN's entered on electronic note-taking devices, uploading handwritten notes scanned into a computer system and any other means for recording notes in electronic form and having them delivered to the patient's record. It is further understood that information systems may be configured to automatically upload PEIE's (e.g. reports) to the patient's PR, such as results of blood work done by a lab that is networked with the EHR. Whether manually or automatically input, all PEIE's are securely uploaded and stored in the patient's PR for later retrieval and review.

Whenever an authorized user needs to retrieve a specific patient's PR, she logs onto the EHR system via, for example, a secure online portal, and searches for the PR in any number of ways. One common way to pull up a patient PR is to enter the patient's first or last name, Patient ID number, date of birth (DOB) or phone number in a search box on the EHR system home page, or “dashboard.” The system then fetches the PR from the EHR database and presents it on the Provider's display, typically in a linear format. Another way, perhaps more useful for smaller EHR's may be to scroll through a hyperlinked listing of all patients in the system that is sorted by some criteria, such as last name, or account creation date.

Conventional graphical user interfaces, or displays, for EHR Provider Dashboards are shown in FIGS. 1A-1C. When a user wants to review the contents of one specific PEIE for one specific patient, hyperlinked text associated with the entry may be displayed on a “dashboard” screen and can be clicked, thereby opening a new screen containing the contents of that PEIE. For example, as seen in the exemplary screenshot of FIG. 1A, an EHR system provided by EnableDoc LLC displays a Provider Dashboard (GUI) 10 for a specific doctor—Dr. Steve Rothschild 1—on staff at a particular healthcare provider. Viewing his dashboard, Dr. Rothschild is presented with an “In-Progress Notes” window 2, displaying in a simple, reverse chronological order a listing all entries for all patients that he has recently seen. The dashboard also presents a Pending Tasks window 4 for all tasks that he needs to complete for his patients. Suppose Dr. Rothschild gets a call from patient Garth Brooks who complains that he is experiencing new pain. In order for Dr. Rothschild to find out what has to be done, he needs to first review Mr. Brooks' patient history, or PR, recorded in this EHR. And, in order to obtain this history, he needs to either enter identifying information for the patient in a search bar such as box 6, such as the patient's name, Patient ID number or date of birth, or he can click on the patients name if the name is readily visible and hyperlinked, such as Mr. Brooks' hyperlinked name 8 is in in-Progress Notes window 2. Clicking on this or any other Garth Brooks link will take Dr. Rothschild to a patient-specific user interface or dashboard for patient Brooks.

Continuing with this example, one user interface that may show up is a Progress Notes (PN) page of the type show in FIG. 1B. As seen from this exemplary patient-specific Progress Notes partial screenshot 20, the doctor is presented with graphical user interface comprising a simple reverse chronologically-ordered listing of Progress Notes for the patient of interest. FIG. 1(b) shows such a typical display for a particular patient having Patient ID #1439. As shown in the viewable screen portion, numerous Progress Notes PN₁ to PNₙ, for Patient #1439 are displayed, showing this patient has been seen by two Provider employees between Nov. 9, 2018 and Oct. 19, 2018, namely, Marci Reiss and Dr. Michael Billauer. In order for Dr. Billauer (or anyone else) to see a particular Progress Note of interest related to a particular Patient Encounter (PE), say PN₂, he would need to click on the hyperlinked box 22 for that note to pull up its full contents (the Nov. 9, 2018 “Return Counseling” PEIE), which will open in a new screen. But as seen here, the “Note Title” entries are not very helpful, as they are all generically titled as one selected from a limited number of options available to the note creator on, for example, a New Progress Note entry form (not shown). A typical set of options might be “Initial Assessment,” “Return Counseling,” “Portal Message” and “Phone Call,” selectable by the note creator in a pull-down menu on the New Progress Note form. Thus, as seen in this exemplary screen shot, even for the short time frame shown on this patient Progress Notes display, if the doctor did not remember the date of the particular PE he was looking for, he would have to pull up (Le. click and view) one Progress Note after another until he finds the Progress Note of interest.

FIG. 1C shows a partial screenshot display of a Notes dashboard (GUI) 26 for a specific patient having a PR in another conventional EHR system in a typical hospital setting. As seen, the dashboard 26 is sorted in reverse chronological order by “Date of Service” for all “Notes” entered by note “Authors” in the system for this patient. This display is even more burdensome to use for finding PN's of interest, particularly when used in a large institution because so many entries that are not of interest to a specific caregiver, such as nurse shift clock-in and clock-out times, clutter the GUI.

Thus, it can be readily appreciated that retrieving a specific PEIE such as a PN of interest from a given patient's PR using conventional EHR GUI's can be difficult, time-consuming and frustrating, especially for with PR's having many PEIE's. This is so because, as seen above, these systems typically display patient record PEIE summary information on screen in a crude line-by-line, linear format, similar to entries in a telephone book, as illustrated in FIGS. 1B and 1C. This makes it difficult to find the content of interest without opening up each hyperlinked line item in this list. Thus, if a patient has a decade of PE's with her Provider, there can be many, even hundreds or thousands, PEIE's, each represented by typically a mere listing indicating the date, the patient's name and a generic description of the type of PE such as a “telephone encounter,” “in person consultation,” “blood test,” etc. In such cases, in order to find a specific PEIE of interest for when a particular patient problem or encounter occurred or was uploaded, every single hyperlinked entry in the patient record GUI display would need to be opened to find the PEIE that was being sought.

To further illustrate this drawback inherent in conventional EHR systems, suppose patient John Doe receives care in “Los Angeles Hospital” which uses EHR System X (EHRSX) and sees both a gastroenterologist and a dermatologist in the LA Hospital system. John's EHR may be opened in the EHRSX by anyone within that hospital system with the appropriate credentials and retrieve John's records that were input by both the gastroenterologist and the dermatologist. However, as the length of John Doe's EHR grows, the ability to retrieve any specific record entry of interest becomes increasingly challenging, because, for the most part, the records are stored in linear chronological fashion. This often requires each hyperlinked line item to be clicked and opened in order to retrieve and view its contents. For example, if one of John's doctors at the hospital needs to see a specific lab result but doesn't know when the lab test was taken and uploaded to the EHRSX, the searching doctor would have to open every lab entry in the linear PR list in order to find the specific results she was looking for. If John had many years of care and many lab result entries in his PR, it may very well result in the searcher simply giving up—not bothering to try to find the record of interest, as it would simply be too cumbersome a task.

Another drawback of conventional EHR systems is their inability to aggregate and present other types of data/information not conventionally presented, but is pertinent to a patient's health, such as research and survey information, in a manner that is both integrated with the patient's medical health record and simple to retrieve and visualize. For example, research data is typically accumulated in case report forms (CRF's), either manually entered in paper forms such as in the CRF 30 shown in FIG. 2A or in electronically-entered forms, such as the Medical History Form 32 shown in FIG. 2B. Once completed, these forms are typically exported into CSV files to be analyzed in statistical analysis programs. Clinical research and survey data may also be collected in Excel spreadsheets, with the data analyzed through Excel macros. Visually, however, it is difficult to look at and make sense of these forms or spreadsheets to find relevant data points or to assess changes without inputting the information into data analysis formats.

For example, suppose a Provider is tracking the reduction in depression for Patient X with Inflammatory Bowel Disease (IBD) over time based on a validated survey instrument called a HADS (Hospital Anxiety and Depression Scale) over a period of two years of treatment. FIG. 3 is a screenshot of one conventional EHR display 40 of patient survey records for a Provider named Texas RCT (Intervention Group) running a study titled “Impact of Psychosocial Care on IBD Patients.” In this example, suppose Patient X is being treated by Texas RTC and is enrolled in this study. Since all entries on this dashboard are presented in linear format, all of Patient X's HADS scores could be viewed by finding and clicking on the “See Survey Results” hyperlink in the “Status” column for each time the HADS survey was taken by Patient X over the course of the two years. As this partial screen shows, there are two entries 42 and 44 having the Survey Title “HADS” with survey enrollment dates Sep. 3, 2018 and Dec. 2, 2018 respectively. Thus, in order to assess changes in the patient's HADS score over two years, a reviewer would have to click these hyperlinks on this page to view Patient X's score for these 2 dates and do the same for all HADS entries on every other page that exist over the relevant time period. It is clear that this effort would be difficult and time-consuming and not easy to analyze HADS changes for Patient X.

Moreover, suppose a researcher wanted to correlate changes in depression scores with other issues the patient was experiencing such as increases in disease activity. To do that conventionally, one would have to open every single HADS score hyperlink in addition to every hyperlink associated with the disease score of interest, such as CDAI PRO2 50 and 52 shown in FIG. 3 to try to evaluate correlations. It would be extremely difficult if not impossible to do this with any meaningful level of continuity and accuracy.

Returning to the proprietary EHR system problem, as was noted, multiple authorized personnel within the same Provider institution or network can enter PEIE's into their patients' PR's. However, since the conventional system used by the Provider is proprietary and closed, no one outside of the Provider's network, such as specialist doctors of a patient who are unaffiliated with the Provider, can remotely access the EHR system to access that patient's PR. Indeed, the unaffiliated medical practice at which the patient's specialist is practicing will likely have it own EHR system—one that does not integrate with or even communicate with the patient's primary healthcare EHR system (such as the hospital system), even if the two systems are provided by the same EHR companies. Thus, as can be readily appreciated, yet another drawback of conventional EHR systems is their inability to share or aggregate patient information from multiple Providers who work in different settings on different systems. Having multiple EHR's for one patient is inefficient, creates opportunities for duplicitous testing and room for error, and makes it impossible for healthcare providers to see the “whole picture” of a patient at one sitting. Indeed, this may be desired by large healthcare Providers—institutions that can use this inefficiency to their advantage—in order to incentive their patients to use only doctors, caregivers and labs that are part of or affiliated with the institution and on the same EHR system. But, this may not be, and often is not, optimal for patients themselves. Since conventional electronic health records reflect the patient encounters only from within the institution using its selected EHR system, PEIE's from Providers working in other places on different EHR systems have no good way to get those PEIE's to the institution. Usually, these records have to be “clumsily” faxed or manually scanned into the patient's records, if at all. Unfortunately, this generally requires human involvement and care coordination, and includes requiring the patient or someone on the medical team to call the other providers or institutions to obtain the records once patient consent for release of records has been obtained. This manual patient record aggregation effort even at its best is tedious, time-consuming, costly and prone to error for even one patient's record, let alone many.

Even worse, if John Doe over the course of a decade saw the gastroenterologist and dermatologist at Los Angeles Hospital on the EHRSX system (say, an EPIC system), but then moved and saw a colorectal surgeon and pain management specialist at San Diego Hospital, which uses EHRSY (say, a Cerner system) as its EHR system, there would be no simple way to merge John Doe's patient data together without someone manually clicking, opening, retrieving, printing, faxing and then manually entering every single line item (or scanning each document) into the other hospital's EHR system.

Even within the same Provider institution, there are often limited merging capabilities. For example, patient John Doe's inpatient records stored in say the EHR system's EPIC platform may not be retrievable by the out-patient clinic providers without the same clicking, opening, retrieving, printing and manual entry by a person responsible for the patient's outpatient records.

Accordingly, what is needed is an integrated EHR system and visual interface, or graphical user interface (GUI), that overcomes the drawbacks of state of the art, widely used electronic health record systems, including the difficulty with using these systems for efficiently finding and retrieving PEIE's of interest.

Also needed is a system that seamlessly aggregates and presents patient data in a manner that allows for the quick visual retrieval of chronological research and survey information pertinent to a patient record.

What is also needed is a robust and comprehensive patient-centric EHR system that is securely accessible to all providers of a patient regardless of where they are, what institutions they are associated with and what proprietary EHR systems they use.

What is further needed is a comprehensive EHR system that records and displays more than just patient records, but also results of surveys, and state of non-medical conditions, such as mental health PEIE's.

### SUMMARY

The present invention meets these needs by disclosing a novel, comprehensive and secure, record storage, retrieval and presentment system, method and interface for easily visualizing and finding Entity Encounter Entries (EEEs) of interest concerning an Entity. The invention discloses an EE retrieval and display system that incorporates a unique visual timeline user interface (GUI) for displaying recorded information. Thus, disclosed is a computer-implemented method for presenting EE information stored in an electronic record data system on the timeline GUI of the present invention. In this system implementation, the Entity Record (ER) is a stored record of all or many encounters of the Entity, the Entity Encounter (EE) is some action related to the Entity, Entity Encounter Information Entry (EEIE) is the full record of a the encounter that is recorded in the ERS, and the Entity Encounter Card (EEC) is a visual card displayed on a screen of a user that contain relevant summary information regarding an encounter of potential interest.

### BRIEF DESCRIPTION OF THE DRAWINGS

Further advantages of the present invention may become apparent to those skilled in the art with the benefit of the following detailed description of the preferred embodiments and upon reference to the accompanying drawings in which:

FIG. 1A is a representation of a partial screenshot of one exemplary conventional EHR dashboard for accessing patient record information displayed on a display;

FIG. 1B is a representation of a screenshot of an exemplary Progress Notes summary page display for a patient over a given time period;

FIG. 1C is a representation of a partial screenshot of another exemplary conventional EHR dashboard for accessing patient record information displayed on a display;

FIG. 2A is a representation of a screenshot of a conventional exemplary case report form (CRF) into which research data may be input;

FIG. 2B is a representation of a conventional exemplary electronic medical history form (MHF) into which patient data may be input;

FIG. 3 is a representation of a screenshot of a summary page created in one EHR system displaying a list of surveys for a study containing research data.

FIG. 4A is a screenshot of one Progress Notes summary page for a patient showing a list of hyperlinked PN entries, in one implementation of the SupportedPatent™ EHR system in accordance with the present invention;

FIG. 4B is a screenshot of a detailed Progress Note corresponding to one of the PN entries in the page shown in FIG. 4A;

FIG. 5A through 5C is a representation of a scrolling display showing a portion of the SupportedPatient™ vertical timeline GUI in one implementation of the present invention for the patient described in FIG. 4A and FIG. 4B, with FIG. 5B a continuation of FIG. 5A and FIG. 5C a continuation of FIG. 5B;

FIGS. 6A-6B is a representation of a scrolling display showing a portion of the SupportedPatient™ vertical timeline, GUI in accordance with another implementation of the present invention, with FIG. 6B a continuation of FIG. 6A and FIG. 6C a continuation of FIG. 6B;

FIG. 7 is a representation of a screenshot of an exemplary completed survey completed by a patient that is accessible via a Patient Health Questionnaire (PHQ) Card similar to the ones shown the visual timeline shown in FIG. 5C and 6B.

FIG. 8 is a representation of a partial screenshot of an exemplary user interface for the web-based Gmail email program;

FIG. 9 is a representation of a partial screenshot of an exemplary inbox view for an email user using the Microsoft Outlook for Mac client.

FIG. 10 is an exemplary screenshot of a display of a conventional email conversation thread related to one of the email summaries shown in FIG. 8.

FIG. 11 is a representation of a screenshot of an exemplary conventional email management panel for accessing email folders or labelled emails, modified with options for user selection of and access to inventive timelines of the present invention.

FIGS. 12A and 12B is a representation of one implementation of a scrolling display showing Email Cards on the vertical timeline GUI of the present invention in an “All Emails” use case.

FIG. 13 is a representation of one implementation of a scrolling display showing the VET GUI of the present invention for an exemplary email conversation.

FIG. 14 is a block diagram showing components of an apparatus in accordance with one embodiment of the present invention,

FIG. 15 is a representation of a scrolling display showing a portion of the vertical timeline GUI in one implementation of the present invention.

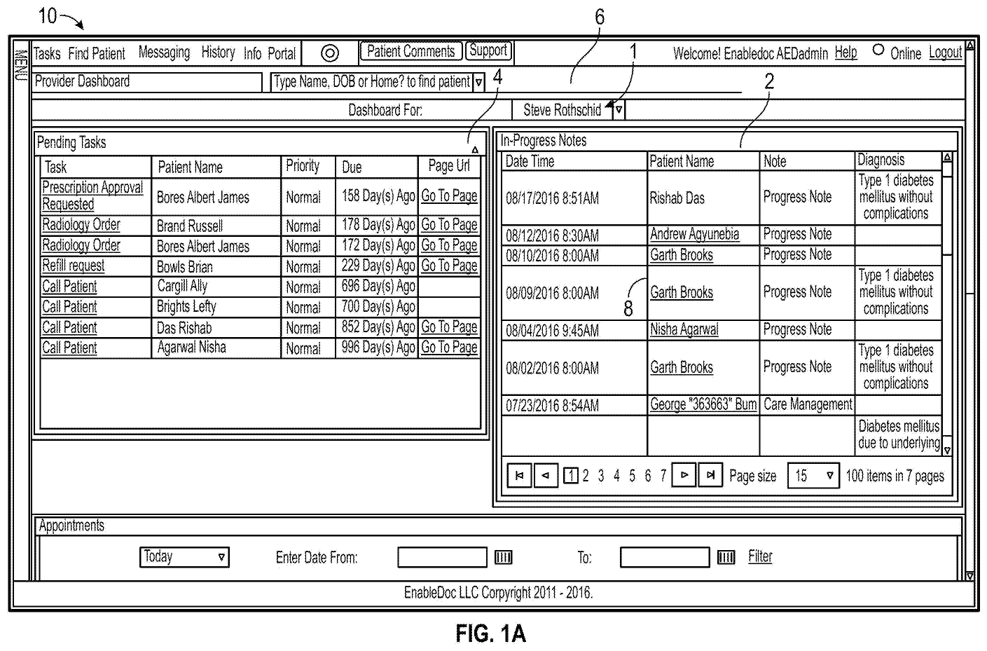

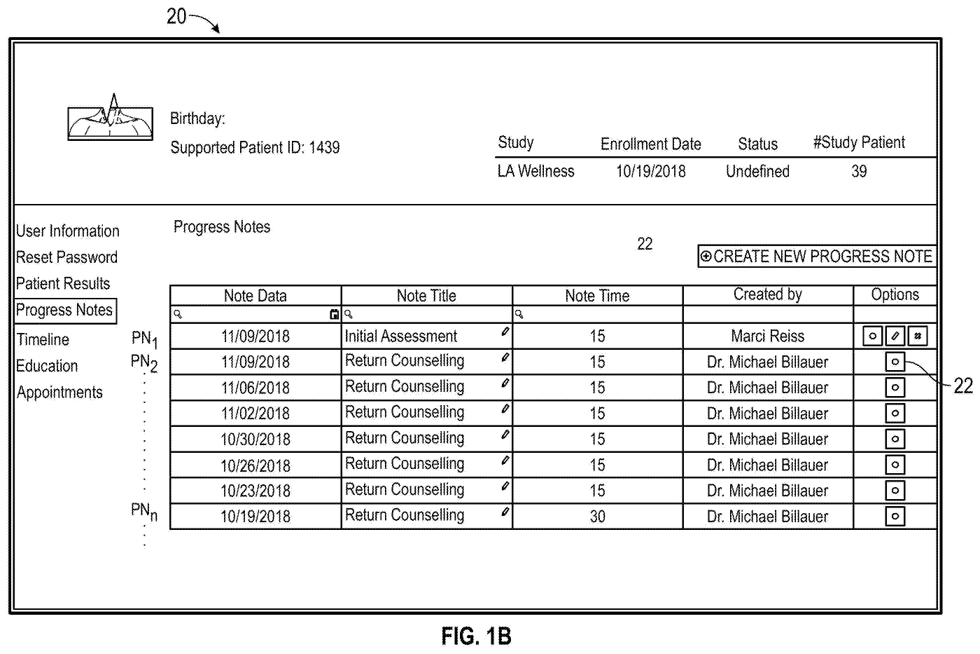

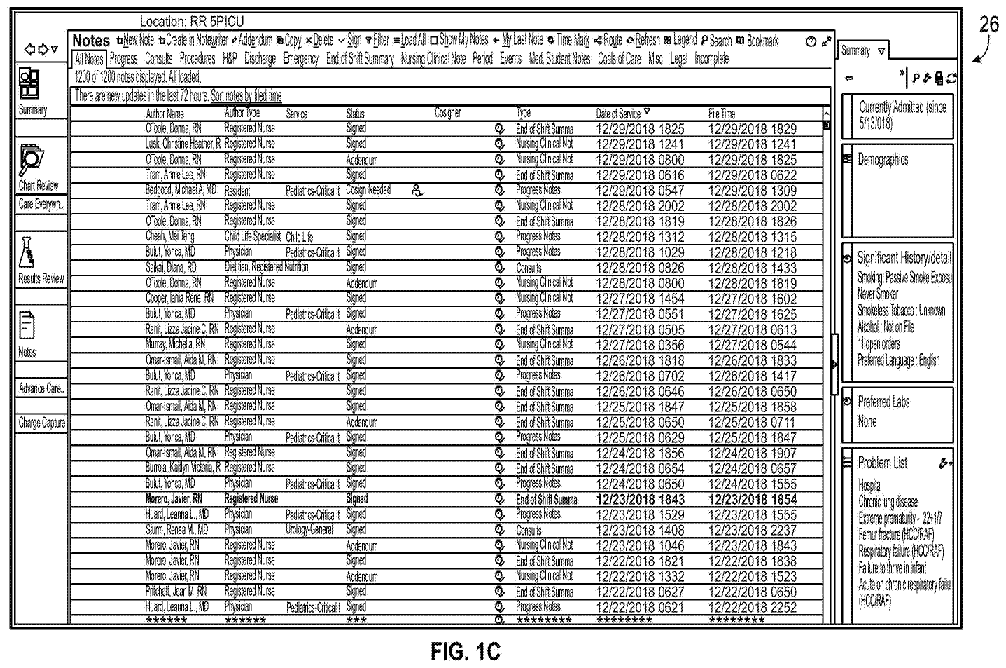

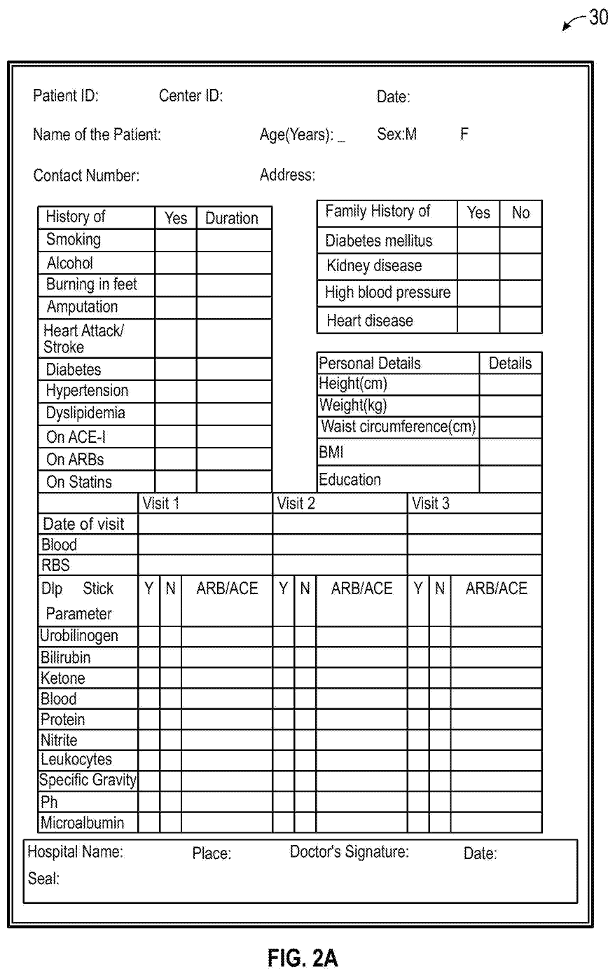

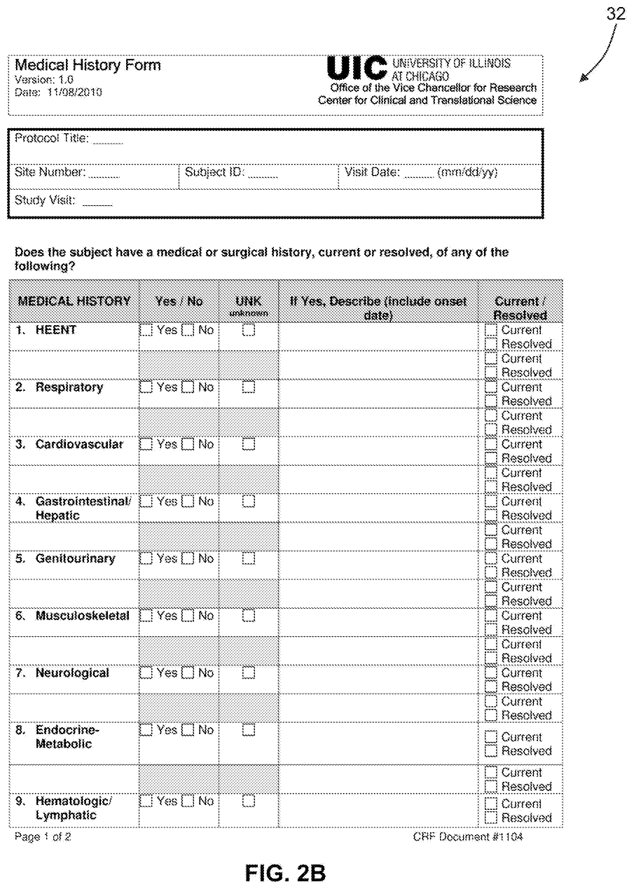

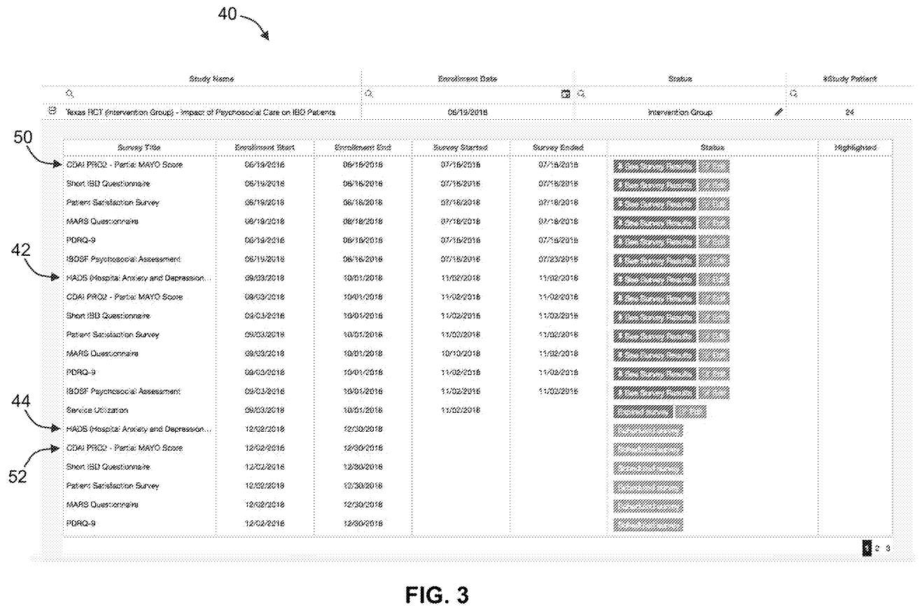

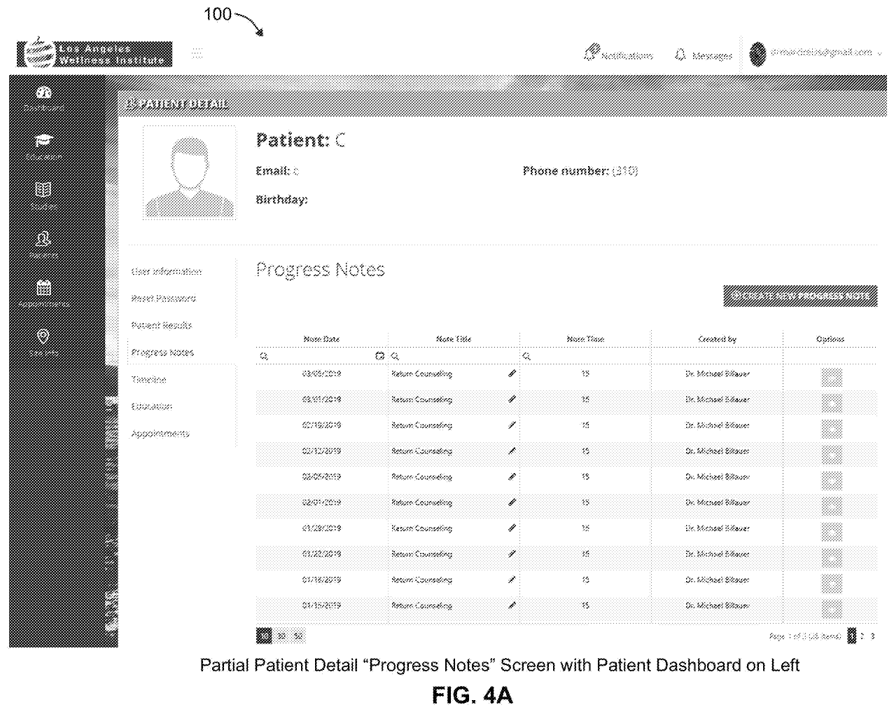

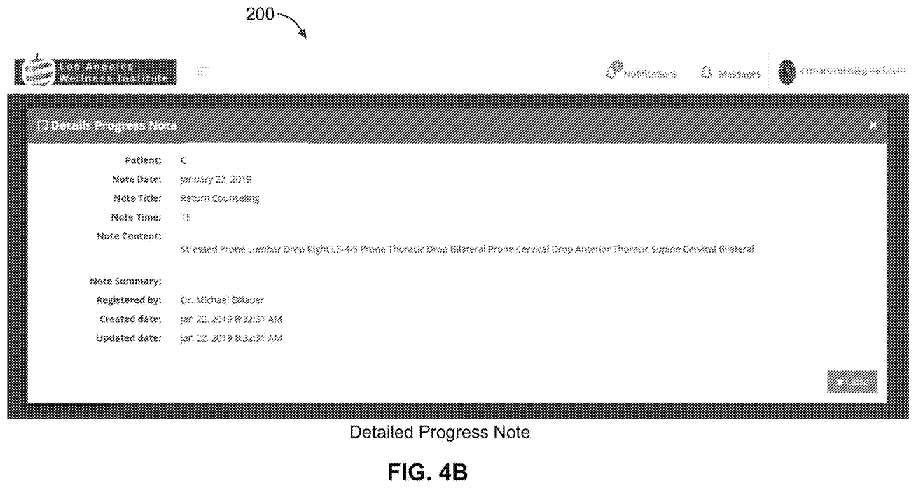

### DETAILED DESCRIPTION OF PREFERRED EMBODIMENTS

Referring now to the drawings, like reference numerals designate identical or corresponding features throughout the several views.

The present invention discloses a novel, comprehensive and, if needed, secure, record storage, retrieval and presentment system, method and interface for easily finding and visualizing electronic record entries of interest concerning an entity regardless of the source of input. The invention discloses a record retrieval and display system that provides a unique visual timeline user interface (GUI) for displaying recorded encounter information concerning an entity. It should be understood that the present invention broadly applies to improve human access to information about any entity that accumulates “encounters” over time. Thus, an entity may be biologic or organic, such as a person, an animal, or plant, or it may be a fictitious entity such a company. An entity may also be any item or system (e.g., physical, electrical, mechanical, etc.) whose state is periodically tracked over time by virtue of encounters it has. As used herein, “encounters” are any occurrences of events or action that may be recorded in a system. Examples of entity encounters when the entity is a person include email communications, patient encounters, customer sales, personal evaluations, etc. Examples of encounters when the entity is not a person may be transactions, events, system performance evaluations, as so on.

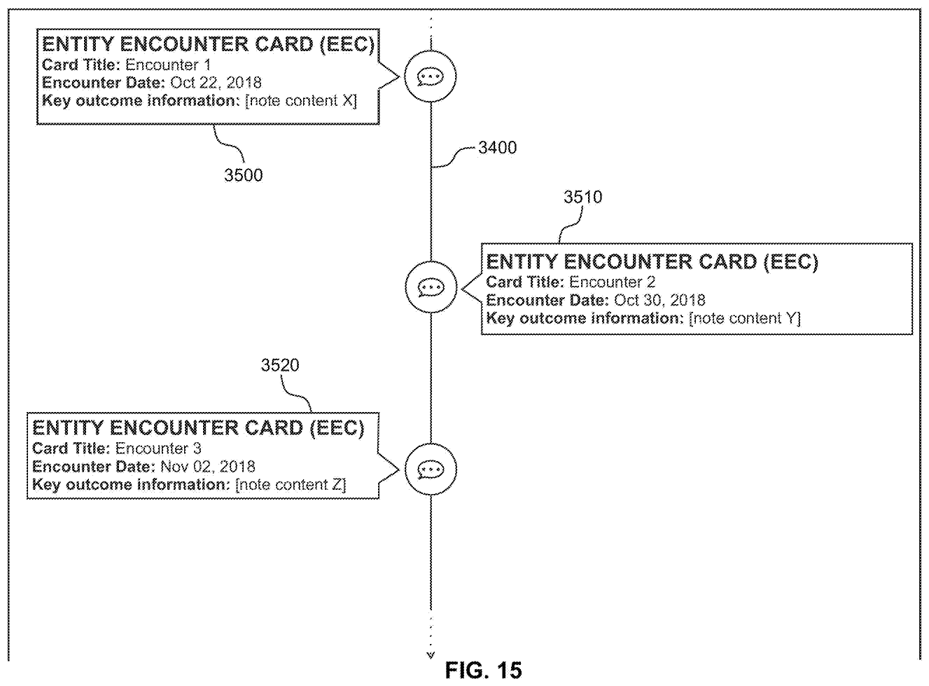

Thus, the present invention discloses a computer-implemented method for presenting electronic record (ER) information for an entity having an Entity Record stored in an ER data system (ERS). As seen in FIG. 15, the method includes displaying on a screen (any computer or mobile or other screen of a user that has access to the novel ERS) Entity Encounter Cards (EEC's) 3500, 3510, 3520 for the entity, each EEC corresponding to an Entity Encounter Information Entry (EEIE) related to an Entity Encounter (EE) with the entity that was electronically input into the entity's ER. Each EEC includes EE content extracted from its corresponding EEIE and, as seen, includes at least (i) key outcome information, (ii) a date for the EE, and (iii) a card title. According to the invention, as also seen the EEC's are displayed on timeline 3400 and are ordered in a chronologically ordered and scrollable timeline for easy visualization and access to relevant encounter information. This way, a user can rapidly view a lot of encounter information in a very short time.

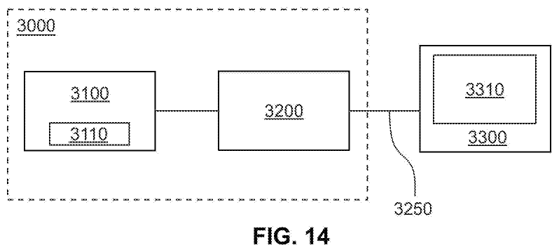

The present invention thus also discloses an apparatus, or system, in accordance with the present invention. In one preferred embodiment, an apparatus 3000 in accordance with the present invention is shown in the block diagram of FIG. 14. As seen, the apparatus 3000 includes a processor 3200, and a memory 3100 that stores computer readable code 3110. This code 3110 when executed by the processor 3200, causes a screen 3300 that is in communication with the apparatus to display a first interface 3310 in accordance with the invention. One exemplary such interface 3310 is shown in FIG. 15. As seen, the first interface comprises a plurality of Entity Encounter Cards (EECs) 3500, 3510, 3520 for a given entity, all on a vertical timeline 3400. Each EEC includes content extracted from an entity encounter information entry (EEIE) recorded in an entity record (ER) for the entity of an electronic record data system.

The present invention has great utility and value in a great number of embodiments. A few will now be presented.

Electronic Health Record Embodiments

One certain set of embodiments of the inventive system and method is disclosed in the context of record-keeping and retrieval of electronic records of healthcare patients.

Thus, specific to electronic health record (EHR) systems, the inventive system provides for each easy input of, retrieval of and access to any Patient Encounter Information Entry (PEIE) for each patient encounter (PE) stored in an EHR system regardless of how many PEIE items there are in a patient record (PR). The system allows for an efficient scrolling of patient information related to their PEIE's with a unique visual display architecture that displays only relevant but sufficient information and content for each recorded PE, dramatically reducing the time and effort needed to find PEIEs of interest, thereby significantly improving clinical efficiency and significantly reducing errors and omissions in retrieving patient records and histories. In applications outside of healthcare, the visual timeline similarly significantly improves work efficiency by significantly reducing the time needed to retrieve records and significantly reducing errors and omissions in retrieving record entries.

As applied to healthcare field, the present invention discloses computer-implemented methods for accessing electronic health record (EHR) information for a patient having a Patient Record stored in an EHR data system. The method comprises displaying on a screen Patient Encounter Cards (PECs) for the patient, each PEC corresponding to a Patient Encounter Information Entry (PEIE) related to a Patient Encounter (PE) with the patient that was electronically input into the patient's EHR. Each PEC includes PE content extracted from its corresponding PEIE comprising at least (i) key outcome information, (ii) a date for the PE; and (iii) a card title. Moreover, the PECs are ordered in either a forward or reverse chronologically ordered, and scrollable timeline that provides authorized viewers with easy and rapid access to relevant PE information.

The PECs can thus provide useful information for any number of patent encounter types and they preferably include links or hyperlinks to its corresponding PEIE such that upon activation the PEIE will be displayed.

PEIEs may comprise records from any type of PE. Thus, PEIE's may be Progress Notes, Lab Results, Patient Procedure Events, Survey Results, or any combination thereof. Accordingly, each PEC on the inventive timeline may comprises any of a Progress Note Card, Lab Result Card, Patient Procedure Event Card and Survey Summary Card, and any other card, each such card corresponding to a respective Progress Note, Lab Result, Patient Procedure Event, Survey Result, or other PEIE.

In preferred embodiments of the method herein, the timeline is displayed as a vertical timeline having nodes representing Patient Encounters (PEs), and wherein each node is visually connected to a PEC. The nodes may contain visual information, such as symbols, indicative of the type of PE represented by its connected PEC. The timeline is preferably dynamically created based on filterable criteria selected by the user, such as PE type that is selectable from pull-down menu available to the user.

The present invention discloses the “SupportedPatient™ Electronic Health Record System (SP-EHR). SP-ERRS is a state-of-the art, EHR system that enables all healthcare providers of any patient anywhere in the world to securely input and securely access all Patient Record information with an ease and efficiency that was heretofore not available. In contrast to conventional provider-centric EHR systems used by most large healthcare system in the market, SP-ERRS is preferably configured as a completely patient-centered EHR system. In particular, SP-EHRS significantly advances the field by enabling any Provider that has a Patient Encounter (PE) to enter or upload PEIE into the patient's record, regardless of who the Provider is, what institution or group the Provider is employed by or affiliated with, wherever that Provider is located.

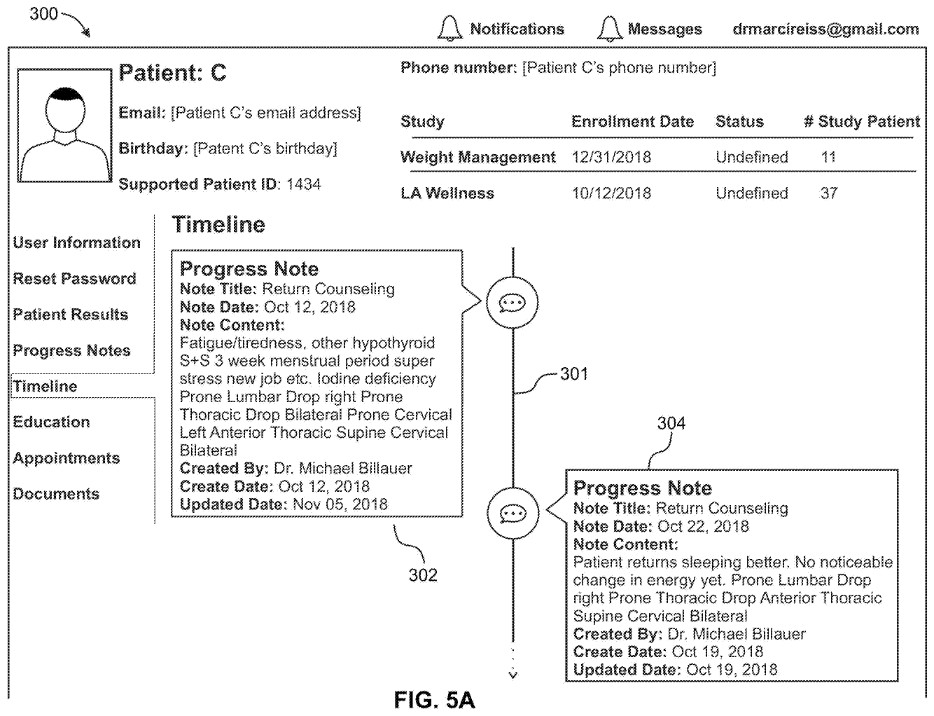

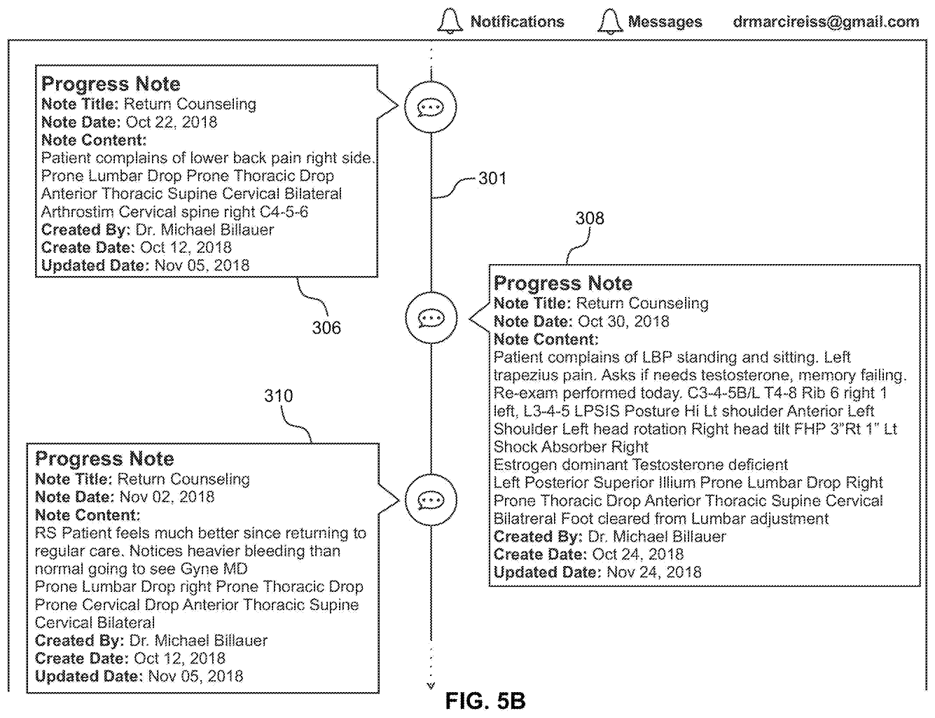

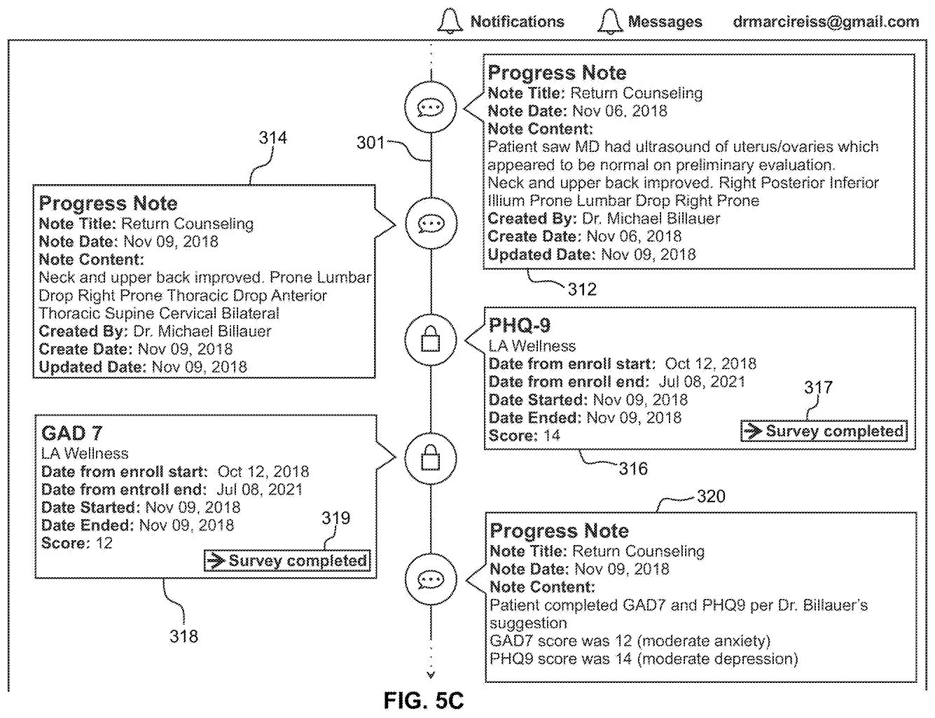

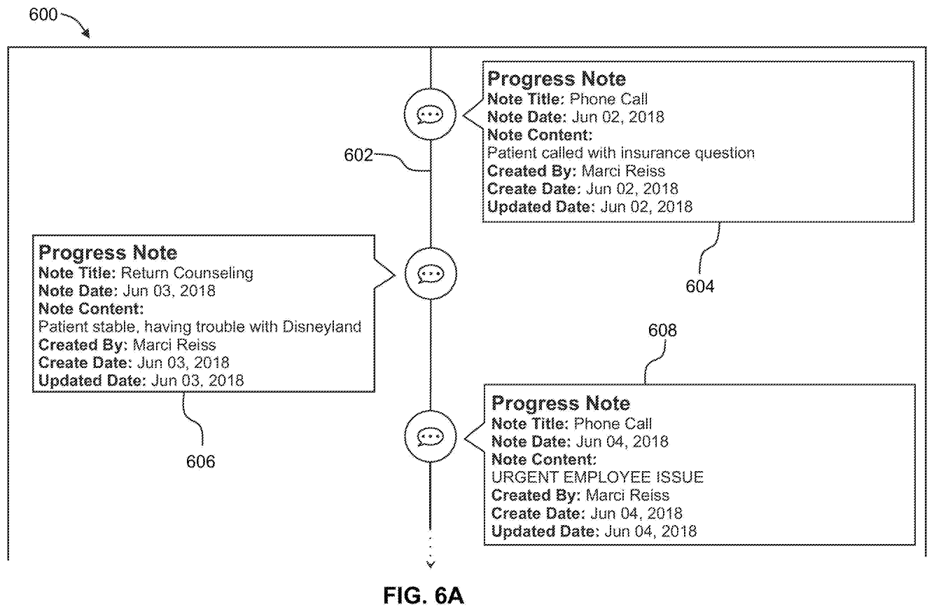

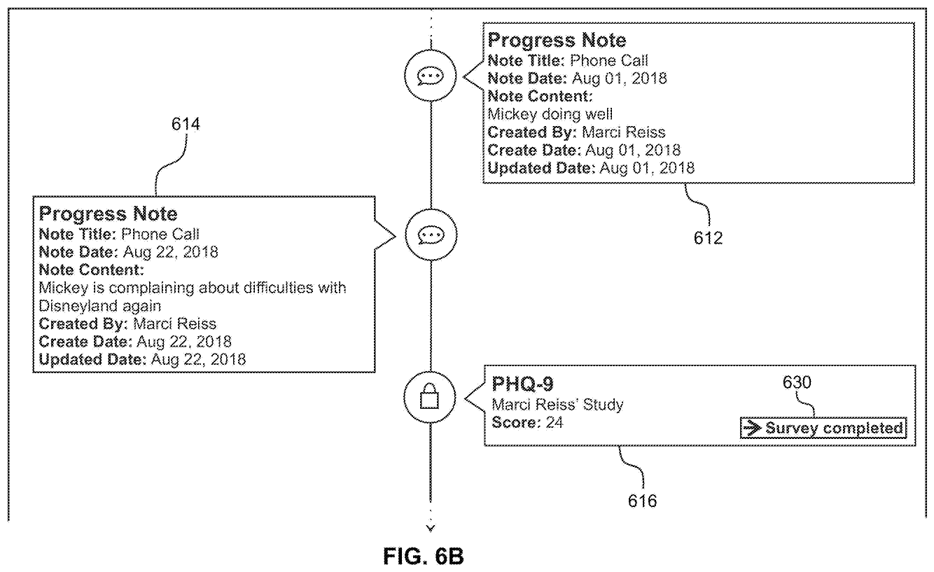

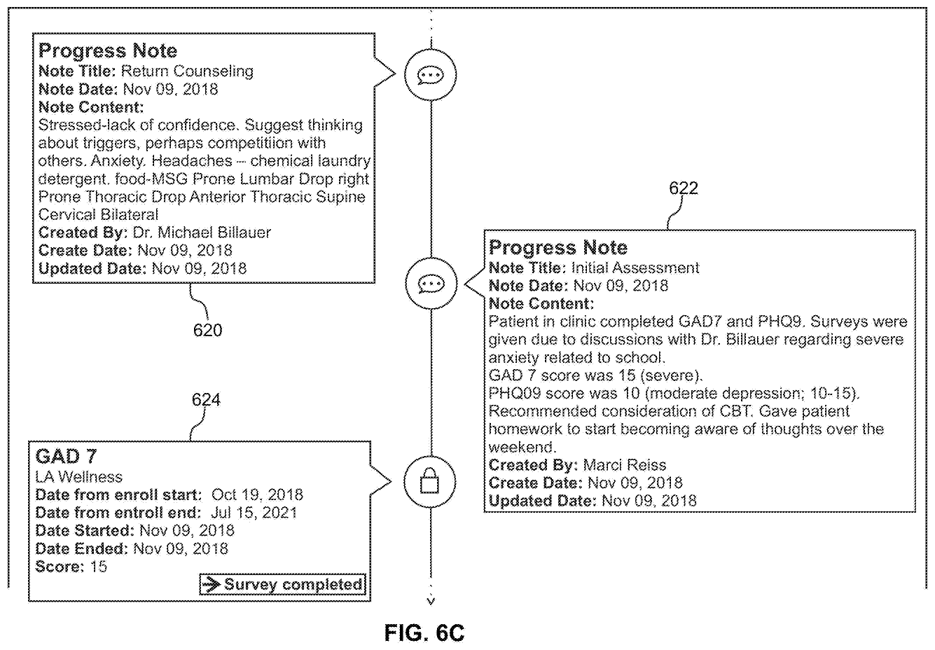

Efficient access to patient data of interest is another important component of the present invention. Thus, the present invention discloses the “SupportedPatient™ Visual Electronic Timeline” (SP-VET) graphical user interface. In the preferred embodiment, the inventive SP-VET is a vertical, scrollable, chronologically-ordered (forward or reverse) timeline having “nodes”, each such node representing a Patient Encounter and having a Patient Encounter Card (PEC) extending from it. FIG. 5(a)-(c) are screenshots showing the GUI of one exemplary partial timeline SP-VET 300 displaying Patient Encounter Cards (PEC's) off the vertical timeline 301 for a patient named “C” having patient ID number 1434. In this embodiment, the innovative upportedPatient™ visual timeline displays chronologically-ordered, patient encounter summary information in the form of “cards” for 10 distinct Patient Encounters, namely PEC's 302-320 presented as nodes on vertical timeline 301. A PEC is a visual display information that takes up less space than an entire visible screen when not scrolling and that displays information extracted from a corresponding full PEIE that was collected and recorded for each Patient Encounter (PE). A PEC may take any suitable shape such as a rectangle, an oval, a bubble or other shape suitable for displaying within its outline encounter information on a screen and that is distinguishes it from other PEC's that may be displayed. Thus, the PEC's displayed in FIGS. 5 and 6, are rectangular-shaped cards. Alternatively, a PEC may not be outlined by any shape. The critical feature of a PEC is that it comprises an organized set of information that is sufficient to enable a viewer (e.g., a reviewing clinician) to know at a glance (a) that it represents a distinct PEIE and (b) whether it represents a PE of interest without having to “click” or hyperlink to the full PEIE record entry itself.

In the preferred embodiment, each PEC will display a minimum of three pieces of information, or fields, namely, (1) a “Card Type” field describing the type encounter the card represents, (2) an “Encounter Date” field, and (3) “Key Outcome” (KO) field. The KO field displays typically summary but sufficient information for the viewer and will depend on the type of PEC presented. Thus, for example, when the PEC is a Progress Note Card, the KO field will display “Written Assessment” or “Note Content” information from the full PEIE Progress Note. In one embodiment, the written assessment comprises a Summary Assessment entered in the PEIE intended to relay the impressions of the doctor or other clinician. In other embodiments, the KO may display the entire PEIE Note Content entered by or for the caregiver. In some instances, PEC's like PNC's will provide all the information a clinician might be looking for and will not have a need to click (link) to the full PEIE at all.

When the PEC is a medical diagnostic result, such as a lab result (blood work) or an imaging result (x-ray, MRI, etc.) the KO displayed may be “Key Findings” that summarizes the results of the diagnostic procedure or test. When the PEC Is a survey, such as a self-reported mental health or other survey, the KO may simply be a survey score (e.g., a number on the depression scale). Moreover, in the preferred embodiment, some or all PEC's may contain links or hyperlinks to its corresponding full PEIE in the patient record enable one to view the full entry for the corresponding encounter.

Back to FIG. 5 and timeline 300, it can be seen that for Patient “C”, from Oct. 12, 2018 through Nov. 9, 2018, eight (8) of the 10 PEC's are PNC's representing patient visits—specifically, one PEC 320 titled “Initial Assessment” and the remaining seven titled “Return Counseling.” The other two are Survey Summary Cards (SSC's), namely, PEC 316 summarizing a PHQ-9 survey taken by Patient C, and PEC 318, summarizing a GAD-7 survey taken by her.

It is understood that additional fields beyond the Card Type, Encounter Date and Key Outcome may be displayed in a PEC. Thus, as in the embodiment shown in FIG. 5, when the PEC is a PNC, seven (7) pieces of information, or display fields from its PEIE, namely: (1) “Card Type”—here, of course, called “Progress Note”; (2) “Note Title”—briefly describing the type of PE; e.g., whether the encounter was an in-person consult, a phone call, an email, etc.; (3) “Note Date”—the date the note was entered; (4) “Note Content” (or Written Assessment field)—the full or partial note describing the PE itself; (5) “Created By” field—identifying the individual who entered the PE entry; (6) “Encounter Date”—here titled, Create Date, and (7) “Update Date”—the date the entry was updated, if at all. It should be understood that all of these fields are exemplary; they will not necessarily be used in all embodiments, and other fields not shown here may be included. However, the inventor of the present invention has determined that these display fields are preferable for implementations of Progress Notes Card.

FIG. 6A-FIG. 6C show another exemplary partial SP-VET 600 displaying 11 Patient Encounter Cards (PEC's) branching off of timeline 602 for a patient named Mickey Mouse.

As can now be appreciated, using this innovative chronological (or reverse-chronological) SP-VET graphical user interface (GUI) enables the user to easily and rapidly locate the PEIE record of interest. This innovative visual timeline structure also enables the user to gain an at-a-glance picture of the patient's medical history by simply scrolling his or her SP-VET display, viewing relevant information for each encounter and through a patient's entire (or partial) EHR. In this way, the time required to find the full PEIE for the PE of interest can be dramatically reduced. When the PEC of interest is found, the user is either presented in the card itself with enough information for her needs, and if not, is able to click inside the hyperlinked card itself or on a hyperlinked word or phrase in the card, and is presented with the full PEIE for that encounter. The vertical PEC timeline format significantly improves clinical efficiency and significantly reduces errors and omissions in retrieving patient records and histories. In fields outside of healthcare, the visual timeline likewise significantly improves work efficiency by significantly reducing the time needed to retrieve records and significantly reduce errors and omissions in retrieving records. It should of course be understood that the innovative GUI of the present invention can be implemented in either an innovative, patient-centric, provider-agnostic EHR system or as an improvement to provider-centric EHR systems to significantly improve the utility of those conventional systems.

As briefly touched on above, the SupportedPatient™ innovative visual timeline can support other types of patient information (PEIE's) in addition to medical Progress Notes relating to an individual's record, that is not conventionally accessible to reviewers in an integrated fashion. In particular, survey results or self-reporting questionnaire results related to a person's mental health state and other test results can be uploaded to the SP-EHRS and displayed in the SP-VET as survey or questionnaire cards Patient Health Questionnaire (PHQ) Cards (PHQC's). For example, PEC 316 and PEC 616 shown in FIG. 5C and FIG. 6B respectively, are displayed in their chronologically correct place in their respective patient timelines along with their PNC's, for easy visual retrieval of the survey results in the context of the whole record stream. Thus, for mental health patient Mickey Mouse, with simple scrolling up and down the vertical timeline 600, one can easily visually see that a Marci Reiss' Patient Health Study concerning Mickey Mouse—namely PHQC 616 titled PHQ-9—was conducted sometime between Aug. 22, 2018—the date of PEC 614 above it—and Sep. 6. 2016—the date of PEC 618 below it, thus chronologically and contextually fitting with other events going on with the patient. As seen, PHQC 616 shows basic information about the study including its name, the start and end dates of the study, and Mickey's score on the survey, namely a score of “14.” See also, PEC 316 shown in FIG. 5C. In a medical research example, if a patient's depression is being measured at specific time points with standardized instruments/surveys, the instrument and access to the questions asked and the results of the surveys will appear in the hyperlinked card, visually interspersed chronologically with the rest of the patient record timeline, easily showing for example, the correlation between changes in anxiety with other medical events going on in the patient's life, as recorded in their patient record.

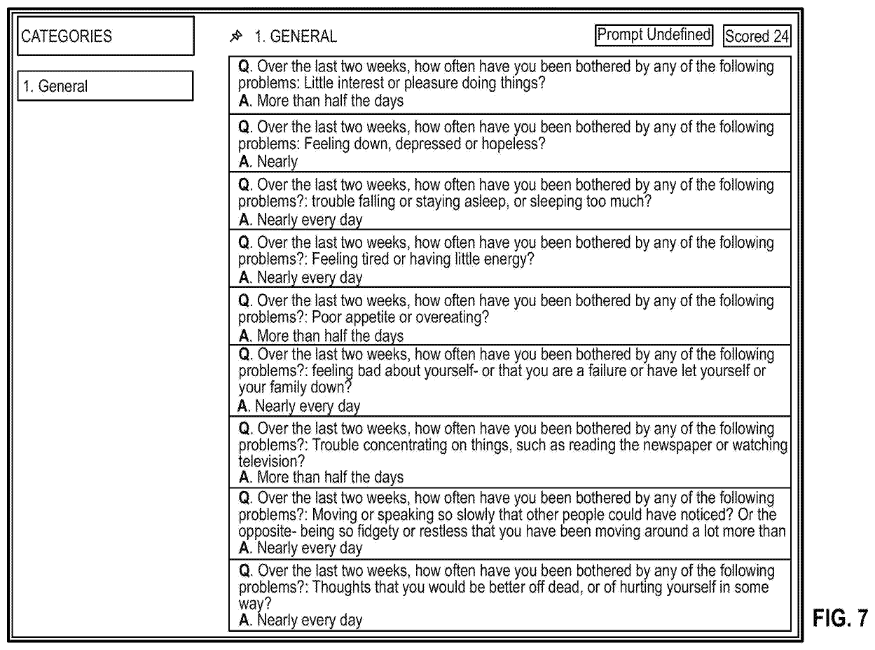

Moreover, the questionnaire that the patient completed is itself is accessible from the comprehensive patient timeline. Thus, clicking on the hyperlinked box 630 inside card 616, titled “Survey completed” will take the user/researcher to the actual survey taken by the patient, in this case, the questionnaire shown in FIG. 7.

In mental healthcare implementations/uses of SP-EHRS, the present invention also has the capacity to send a “Prompt” to a Provider if the patient answers any question on any survey a particular way. For example, as seen in FIG. 7 on the PHQ-9 form, there is a single question about suicidality (the last question). Providers have the ability to set up their SP-EHRS dashboard so that if any patient indicates that they are suicidal on that question of that survey at any point, they will be immediately notified using conventional critical event notification technology, as will be understood by one of skill in the art

Progress Note Cards PNC's can be optionally set up to allow for significantly more fulsome Note Content than those shown in the PNC's shown in FIGS. 5 and 6. For example, as seen in Timeline 600, in an implementation which focuses on patient mental health care, PEC 622 shows notes with a substantial amount of content and reporting of the GAD 7 and PHQ9 scores. In such embodiment, the vertical timeline of the present invention may provide the provider/reviewer with sufficient “at-a-glance” information, obviating the need for reviewer to click on these hyperlinked cards for the source or additional information.

An example demonstrating some of the unique benefits and centralizing power of the comprehensive, cross-discipline innovative SP-EHR system of the present invention is now described. 32-year-old John Doe presents to the Dream Inflammatory Bowel Disease Center (DIBDC) in January, 2018 with persistent abdominal pain, a 15-pound weight loss, perianal pain and anemia. After a thorough evaluation including blood work, stool and imaging studies, he is diagnosed with small bowel Crohn's disease. The DIBDC opens a patient record for John in the SupportedPatient™ EHR system and the doctors attending to him enter PEIE's in his electronic record. But John is also very anxious as he is concerned about the impact of his new diagnosis on his ability to perform his duties at his job and his ability to care for his 4-year old and 2-year old children. His anxiety is overwhelming him and so his gastroenterologist G at DIBDC refers him to therapist T for counseling.

After an evaluation and several therapy sessions, the therapist refers him to psychiatrist P for medication management of his anxiety. Additionally, due to the discovery of a perianal fistula, John is referred to a colorectal surgeon CS for drainage of the fistula.

John is also referred to a nutritionist N to help him regain weight. Subsequent to his surgery, John is referred to pain management expert PME to help him wean off the opiates that he was give post-operatively. The records that John accumulates in his 1st year post-diagnosis are extensive, including the 6 providers G, T, P, CS, N and PME.

Over the following 10 years, John's patient record continues to grow with the addition of other providers include a primary care physician PCP. John needs to switch to a new gastroenterologists G2 during those years due to his main gastroenterologist G moving away. He also switches to a new nutritionist N2 due to the later development of short bowel syndrome subsequent to another two surgeries. During the 10 years since diagnosis, John has also gotten a divorce, which led to an episode of major depression. John used many different medications for aggressive Crohn's disease and due to his multiple disease flares, he is enrolled in a clinical trial of a new biologic therapy that is in Phase III and is showing a great deal of promise. John's disease does not improve and due to extensive inflammatory based pain, John has developed an opiate addiction.

Over these 10 years, John's EHR data is extensive with countless encounters with each of his 9 different healthcare providers. The primary care and gastroenterologist work in the same hospital with the surgeon. However, the psychiatrist, therapist and pain management expert work at unrelated facilities. The nutritionist runs her own private practice and is not connected to either of the other medical sites where John is receiving his care,

In the conventional model with no centralized electronic health record system, it can be appreciated that there exists no good way to aggregate the data and records that John has accumulated over the ten-year period. Since the different facilities use different electronic health record systems, there is no realistic way for the aggregation of that data. In John's case, his records from his gastroenterologist and primary care provider are recorded in, say, EPIC. His records from his psychiatrist, therapist and pain management expert are recorded in CERNER. The nutritionist has her notes in a small practice EHR system. The clinical trial records are recorded in a dedicated clinical trial Oracle database set up by the pharmaceutical company. It is conceivable but highly improbable and unrealistic for any one person or service to aggregate all the data into one system. Even if there were such a person, the likelihood for error in the data aggregation process in such an endeavor is incredibly high.

The present invention also solves this significant problem. This is illustrated by the following fictitious example. As part of the U.S. government's determination to help opiate patients and stem the national crisis, the government has established the National Opiate Cessation Center (NOCC). The idea is that all providers managing chronic opiate disease patients regardless of location would be registered and embedded into the NOCC, allowing for both the creation of a nationwide patient record for each patient and the secure aggregation and appropriate sharing of PEIE's from patients across the country, in spite of the patients' providers working in different institutions and medical settings on different EHR systems. NOCC selected SupportedPatient™ as its comprehensive EHR System. Now, all record entries can be presented to all Providers in one streamlined visual chronological display of information, easily read through by scrolling down through the patient entries.

Through the action of a simple scrolling down through the record, the patient's PE's from all of the providers from primary care and gastroenterology to pain management and psychiatry, to nutrition, surgery and research, can all be visually seen, even though the providers work in different settings. Additionally, there is no need to open individual PEIE's as all of it can be easily viewed through the chronological timeline.

Email Embodiments

The present invention also has great utility for improved email management. Email has become a ubiquitous and indispensable means for electronically communicating information between and among people. Popular systems include web-based email such as Google's Gmail, Yahoo! Mail, and Outlook.com, and client-server email systems often used in the workplace. The latter includes POP3, IMAP and MAPI email servers that centrally manage email traffic from and to client applications on email users' devices. One system used by many businesses, for example, is the Microsoft Exchange Server (often called “Exchange”). All email server-based systems serve up emails sent through them to any of myriad client-side applications used by users with credentials to the server the system. Examples of client email programs include Microsoft Outlook for desktops or Mac, and a host of apps for mobile devices, such as the native iOS Mail app for iPhones and many different mail apps for Android devices.

In addition to enabling users to compose and send emails, all user-side software—whether web email or an email client (software)—provide user interfaces with the same basic functionality—they all (a) can display, in defined areas of a screen, email summary information for each email received by and sent by the user, (b) enable the user to select (click on) any of the visible email summary areas to pull up (or, hyperlink to) the full email corresponding to the summary information, and (c) offer varying degrees and methods of management of sent and received emails. The typical email client thus has an “inbox” (some call it an “inbox folder”), into which all incoming email is initially sent and stored, a “sent folder” that shows emails sent by the client to others, and usually other basic folders like “draft” folder, the “deleted” or “trash” folder and so forth.

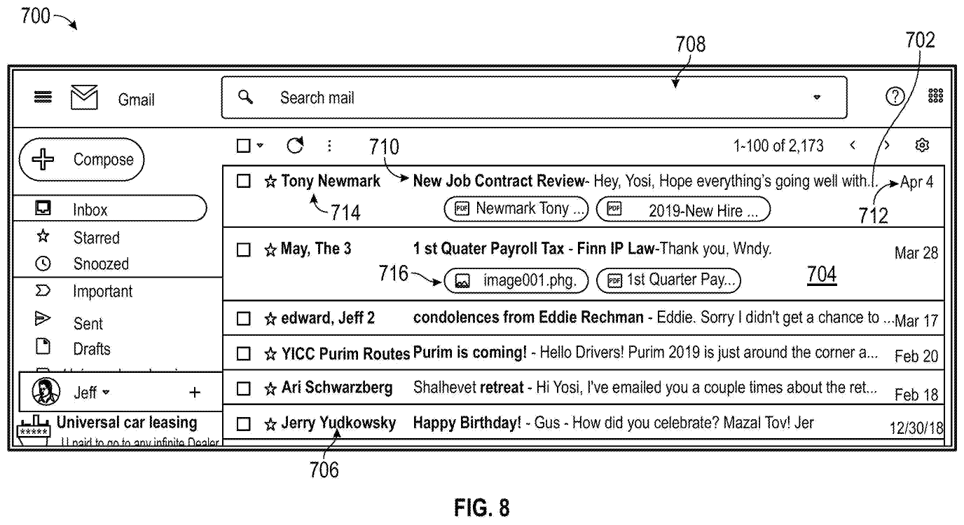

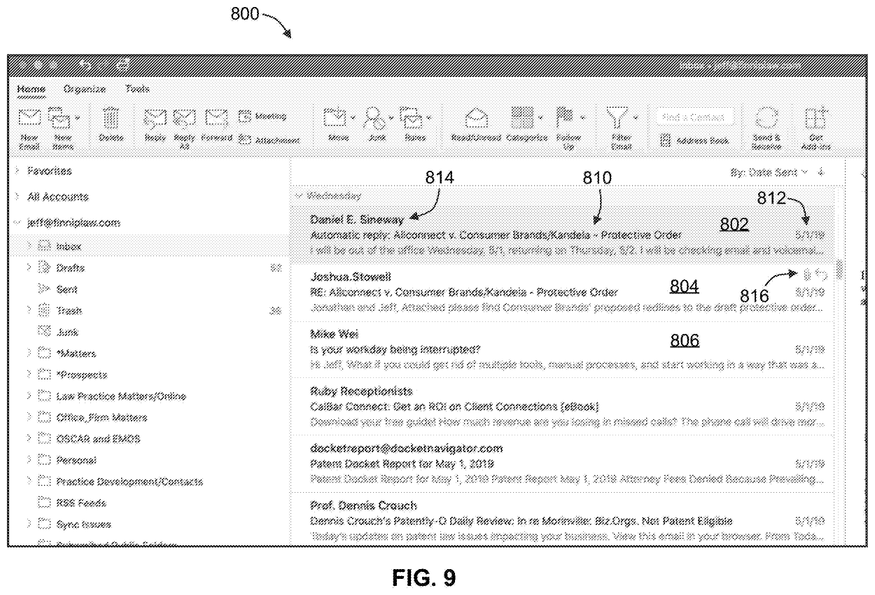

By default, the email summary information displayed for each email in a conventional email client application folder such as the inbox comprises a combination of viewable email metadata and some portion of the body, or content, of the email itself. While the specific summary information displayed for each email will vary from app to app, they all usually include basic visible metadata (information about the email) and some portion of the body of the email. Examples of such summary information shown for conventional folders such as the user inbox, are shown in the partial email user interfaces shown in FIGS. 8 and 9. FIG. 8 is a partial screenshot of an exemplary user GUI 700 for the web-based Gmail email program, showing an inbox view, and FIG. 9 is a partial screenshot of a GUI 800 displayed for an inbox view of an email user, jeff@finniplaw.com, using the “Microsoft Outlook for Mac” client. As seen in these figures, the metadata that is displayed includes: a “Subject” 710, 810 (up to certain number of characters), the “Sent Date” 712, 812, who the email is “From” 714, 814 (in the case of the inbox) or “To” (in the case of the sent folder), and a symbol 716, 816 indicating whether or not there is an “Attachment”, respectively. These defined email summary areas are contained in defined regions on the screen, such as rectangular boxes 702/802, 704/804 and 706/806 respectively, and are presented linearly, one on top of the other in row-like format, and usually in chronological order (usually most recent at the top). Some email programs allow the user to customize the view. For example, some allow the user to change the size of the defined email summary areas, but any changes to will apply uniformly to all email summary areas in the folder, Some also allow for customizing the order of presenting the list of summaries in the folder (inbox, sent folder or other). It is understood that many other features are available in many conventional email clients and web interfaces available to the public.

As is well understood, the defined email summary areas are hyperlinked, or contain hyperlinks, to their corresponding full emails. Thus, when a user is reviewing emails in her inbox, she can scroll down (or up) the inbox GUI to review rows of email summary information. When she finds an email summary of interest, in order to see the full email and if needed take some action on it (e.g., compose a reply or forward email, or open an attachment), she can pull it up by clicking in the hyperlinked defined summary area with a mouse (on a computer display) or, in the case of a mobile app, by touching the email summary area, thereby causing the program to display the full email, often in a new window. For any given email interface, the same basic GUI and email summary structure discussed here is used for all predefined “folders” provided, such as the “sent,” “draft” and “deleted” or “trash” folders, as well as for user-created folder.

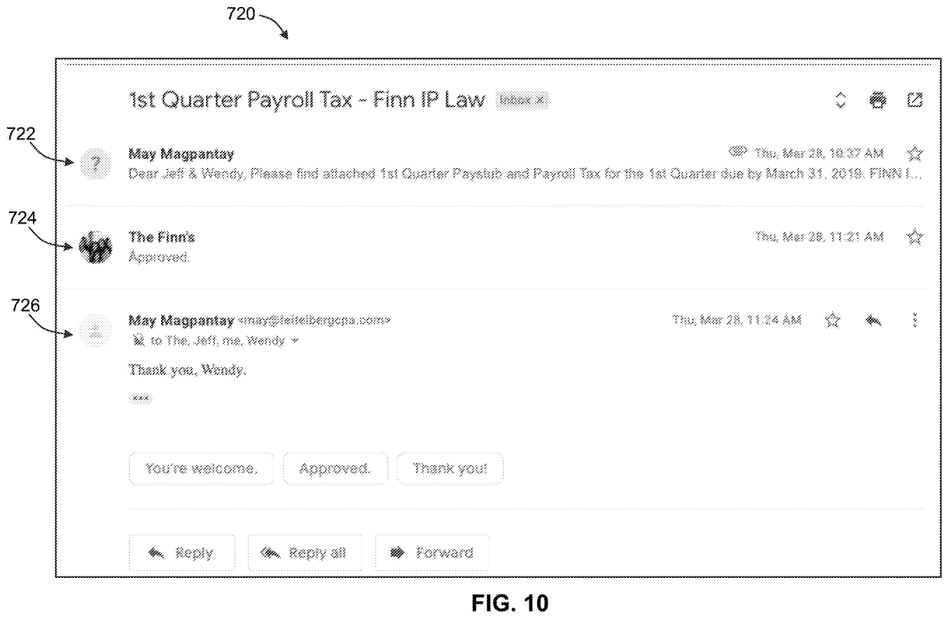

Moreover, when one does find and select an email of interest from a folder in order to view the full content of the email, that email may or may not be a response (reply) to another email, or even one email in a chain, or thread, of many emails comprising a “conversation” or “conversation thread.” Thus, when the email of interest is part of such a conversation, selecting it will often pull up a window showing the entire thread of emails—the full conversation—whether the user is interested in seeing the entire thread or not. An example of this is shown in FIG. 10. In particular, when the user clicks on email summary box 704 shown in FIG. 8, as seen in FIG. 10, the entire email chain connected to that email or “conversation” 720 is displayed. In this case, there are three (3) emails in this conversation: email 722, the first email in this email chain, email 724—the reply to email 722, and email 726, the reply to email 724, which is the email corresponding to email summary 704 in FIG. 8. Since this email thread has only 3 emails, the user's computer screen may be large enough to show all three emails at a glance, as shown in FIG. 10. Oftentimes, however, there are many emails in one conversation thread, such that one will need to scroll up or down the email conversation thread screen in order to read all emails in the conversation or to find the one email in the chain that is of interest.

Because the volume of received and sent emails for many users is so large, and many people wish to keep many emails they receive and send (as opposing to deleting them right away), email systems need to offer email management tools to help their users manage them. Indeed, users today often keep thousands or even tens of thousands of incoming and sent emails in their systems. Thus, having ways to easily and rapidly find emails has become a critical function of email clients. To address this, conventional email clients typically provide some combination of search capability and user-created folders. Searching is usually enabled by providing somewhere on the interface a search box, such as search box 708 in FIG. 8, that enables the user to type in a term or name, or date in order to pull up all emails meeting the search criteria residing in a certain folder (like the inbox) or the entire email account of the user. Some interfaces offer basic or advanced filtering capability to enable the user to limit the searching to emails meeting selected criteria (such as date range, only email “to” a certain email address, search only certain folders, etc.). Most commonly, most email systems enable users to create and label as many folders (or appended labels to emails) as they wish, as well as folder within folders (“subfolders” or “nested-labels”) into which they can move emails from their inbox or sent folders (or other folders) into those folders. This way, for example, all emails relating a certain client, person or matter can be stored or found together in a meaningfully-labelled folder (or tagged with a common label), for later summary review and/or searching.

Indeed, a great deal of effort has been expended by email system providers to improve email management. Unfortunately, however, all conventional graphical user interfaces come up short in their ability to easily manage, search, find and review communication to and from different senders. When a user wants to review the contents of one specific email in their inbox or any other folder, he must often click on the hyperlinked text associated with the entry to open its full contents. To retrieve the full content of the email, the hyperlinked summary needs to be choked. To find a specific email, say in an Inbox with hundreds, thousands or more emails, one may best be served by entering key search terms into the search box. But if key terms are found in many emails stored in the Inbox, they will be all displayed in a similar manner to FIGS. 8 and 9, which may not be helpful.

Moreover, when a hyperlink is clicked, if there are many emails in the single conversation thread or conversation between the sender and receiver (or among many email users), even in the expanded view, the existing client GUI's do not provide a clear way to visualize the entire conversation, or to find specific emails within the conversation.

It can be readily appreciated that there are significant drawbacks and difficulties with finding emails or specific parts of email conversations stored in a users' accounts using conventional email GUI's. This retrieval can be difficult, time-consuming and frustrating, especially for individuals with extensive email histories. Because the displays in conventional email systems are displayed on the screen in a crude line-by-line, linear format, it is difficult to find the content of interest without opening up each hyperlinked line item in this list. If an individual has years of emails, this is particularly challenging and frustrating. These same frustrations and challenges occur with different categorized subfolders including “Sent” items, “Starred” items, “Draft” items, or other items sub-categorized in the current conventional systems.

Thus, the system and method include displaying on a screen Email Cards (EC's) for a user on the timeline, each EC corresponding to an email related to the user that was sent by or received by the user. Each EC includes content extracted from its corresponding email and includes at least some metadata and email body content. According to one embodiment of the invention, the displayed EC's are ordered in a chronologically-ordered and scrollable timeline for easy visualization and access to email information, simplifying and speeding up the visualization and finding of communications of interest.

Thus, disclosed is a computer-implemented method for presenting email information from emails associated with a first email user account of an email system. The method comprises displaying on a user screen Email Cards (ECs), each EC corresponding to an email stored for the first user account. Each EC includes email content or data extracted from its corresponding. The content comprises at least a date, a timestamp or both identifying when the email was sent or received, a card title, and content from the body of the email. The EC's are displayed on a chronologically-ordered timeline. Moreover, the timeline is preferably displayed as a vertical and scrollable timeline and each EC is visually connected to the timeline. It is understood however, that the timeline may be displayed in another orientation, such as on a horizontal timeline, or other orientiation that, for example, may be most user friendly on the display of a mobile device.

The ECs generated and displayed on the timeline may correspond to all emails stored in the user's email account or less than all, such as those stored in a selected folder, or labeled with a given label. The selected folder may be any of the user's inbox, a sent folder or a user-created folder. In one embodiment, at least one EC further includes a first link area containing a link or hyperlink to its corresponding email such that upon activation causes the email to be displayed. In another, at least one EC contains information identifying the number of emails associated with the email represented by the EC. The association comprises an “email conversation” between or among the first user and one or more additional email users in email communication with the first user.

The ECs may further include an area having a link or hyperlink that when activated causes a new display of a timeline of ECs associated with all emails in the conversation to be displayed. In one embodiment, the timeline is dynamically created based on filterable criteria selected by the user. The filterable criteria may be anything relevant for the user, such as a “date range”, “sent to” or whether or not there are attachments, to name a few. In one embodiment, the ECs are ordered in a forward chronologically-ordered timeline, and in another they are ordered in a reverse chronologically-ordered timeline.

The present invention may further include the step before the displaying step, of receiving a selection from a user of the email system indicating a request for said timeline of ECs to be displayed, wherein the selection from the user is made from an add-on to an existing email system.

The present invention also discloses an apparatus that includes a processor and a memory, the memory storing computer readable code that, when executed by the processor, causes the processor to display a first interface, the first interface comprising a plurality of email cards (ECs) corresponding to emails stored for a first user having an email user account of an email system, each EC including content extracted from its corresponding email, and wherein the plurality of ECs is ordered chronologically on a scrollable vertical timeline displayed on a screen.

In one embodiment, at least a portion of each of at least some of the plurality of ECs is selectable such that selection of the portion of one of the selectable ECs causes a second timeline to be displayed comprising information from the email corresponding to the selected EC.

The present invention discloses a revolutionary method for a user to access, view and manage emails in an email account by disclosing and implementing the novel visual timeline user interface of the present invention as a “Visual Email Timeline” (VET) graphical user interface. In the preferred embodiment, the inventive VET generated by the GUI software of the present invention provides a vertical, scrollable, chronologically-ordered timeline having “nodes”, with each node representing an email communication having an Email Card (EC) extending from ft, EC's are visual “cards” containing email metadata and email body summary information or full email information that is more useful and easily digestible for a user than the conventional row-based email summary entry GUI's available in the prior art.

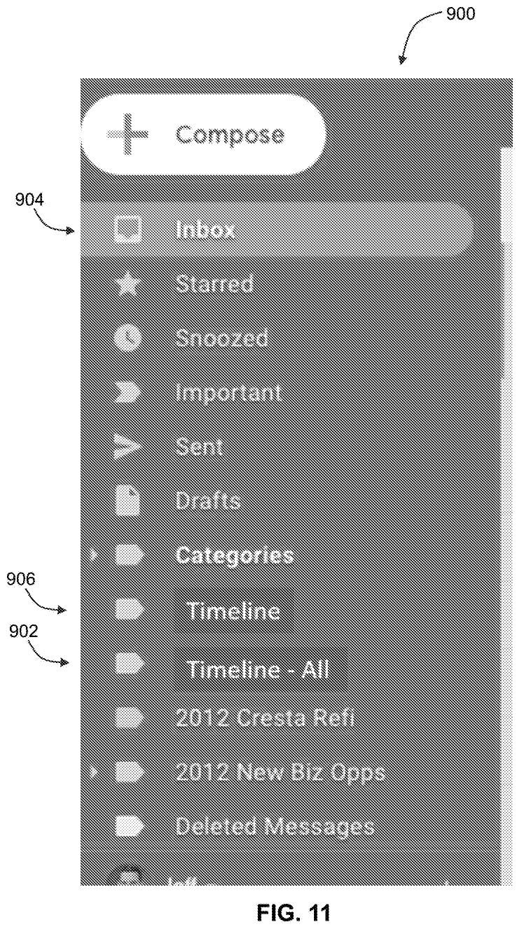

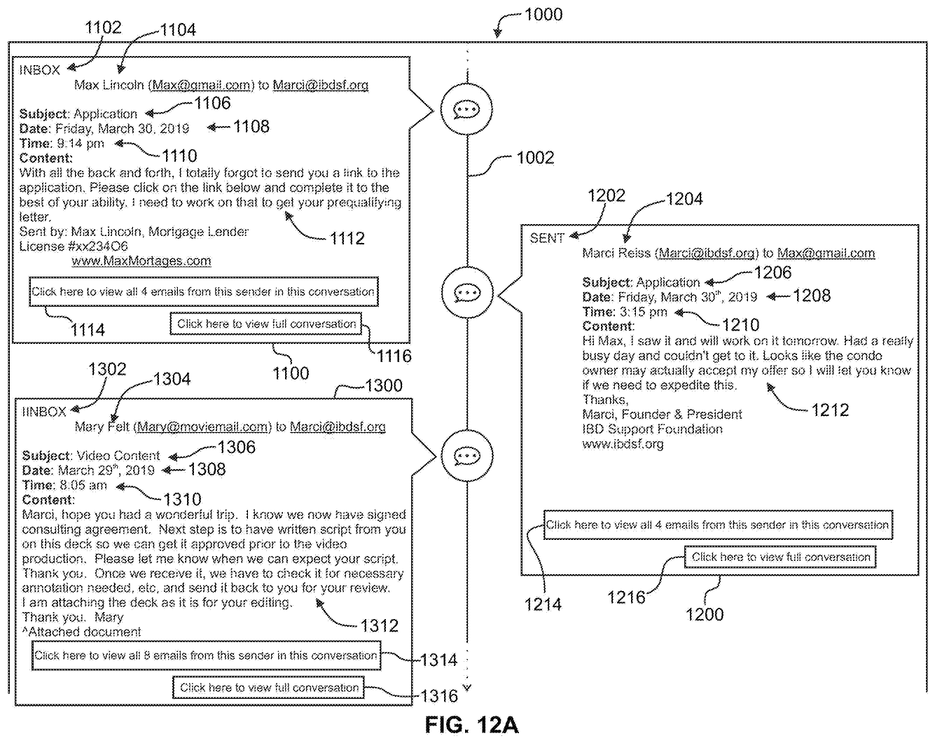

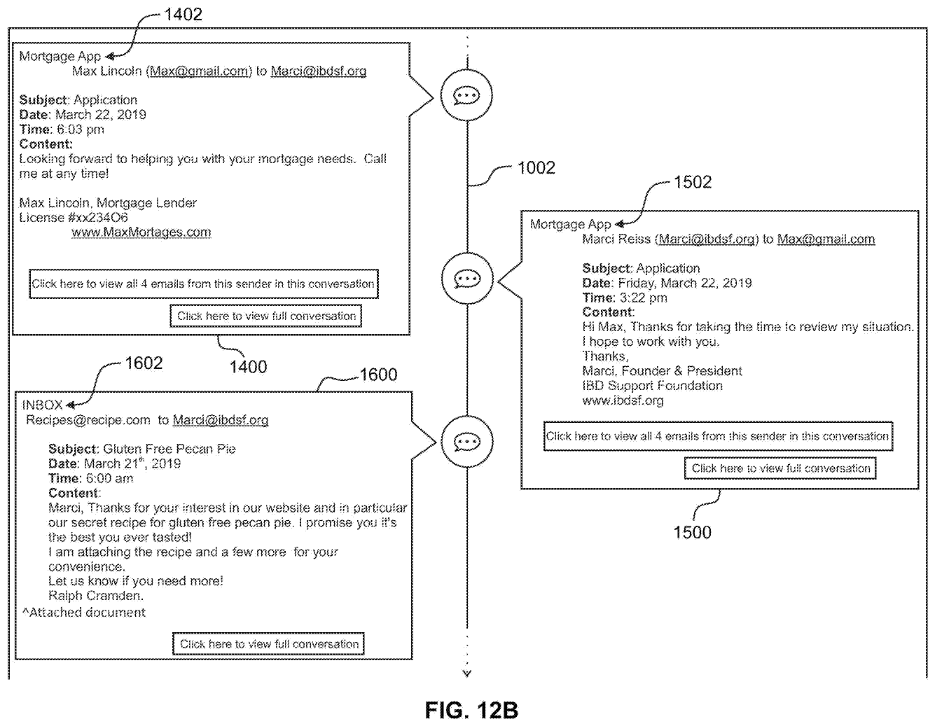

FIGS. 12A-B show one exemplary representation of a first embodiment of the VET of the present invention. These figures show a partial “all-email” VET 1000, as may be dynamically generated on a computer screen when a user first opens his/her email application or web interface. Alternatively, VET 1000 may be generated upon selecting an “all-email” VET option, made available on, or in connection with, a conventional email client via a hyperlink. This may be presented in an email client management panel, such as panel 900 shown in FIG. 11. In such case, when a user clicks the hyperlinked term “Timeline-All” 902 (or symbol or any other representation as will be understood by one skilled in the art), the VET software generates VET 1000 shown in FIGS. 12B and 12B, generating on the vertical timeline bar 1002, in reverse chronological order, all Email Cards (EC's) 1100-1600 (in hundreds increments). Each EC is associated with or visually connected to (although not necessarily with an actual connecting line), and is placed at either side of, vertical line 1002. In the preferred embodiment, the EC's are placed on alternate, left and right, sides of line 1002.

Turning back to FIG. 11, the invention also contemplates the “at a click” ability to generate a VET of the presently displayed folder of (or, labeled) emails of existing email programs. Thus, as seen in panel 900, the inbox is currently highlighted at 904 indicating that the inbox is being displayed. Clicking on the option Timeline 906 would then generate a chronological VET for all emails stored (or associated with) in the inbox of the client program.

Because of the user friendly, simple reviewable and scrollable structure of the timeline, each dynamically generated EC can be loaded with email information. The cards may include summary information or may be programmed to provide all information visible in its corresponding full email, effectively serving as a re-rendering of the email as would be seen in its “native” format. Thus, as shown in FIG. 12A, cards 1100, 1200 and 1300 each includes a field for displaying the folder the email is in or the label appended to the email, namely, INBOX 1102, SENT 1202, and INBOX 1302 respectively. The cards also include the following email metadata fields: “Author” of the email 1104, 1204 and 1304, respectively, “Subject” field 1106, 1206 and 1306, respectively, “Date” field 1108, 1208 and 1308, respectively, and “Time” field 1110, 1210 and 1310, respectively. In this embodiment, as seen, the full contents of the body of the emails 1112, 1212 and 1312 are also provided in these EC's. It will be understood that displaying the entire email body regardless of the length of its message can cause the dynamically-generated cards on any timeline to vary in size. In an alternative embodiment, the system may be programmed to limit the size of the email body content display field to a fixed or a maximum number of characters (or words). In this way, all cards can be made of uniform size.

As further seen in FIG. 12A, ECs also optionally include some additional “at a glance” information. First, notice boxes 1114, 1214 and 1314 may be included that display for the user the total number of emails sent by the sender of the EC email in the related conversation. In the present embodiment, these boxes are hyperlinked such that upon selection, they will dynamically create new VET timelines showing only the emails in the conversation from (or to) the sender. Also, hyperlinked boxes 1116, 1216 and 1316, respectively, are provided to enable the user to generate a new VET display that displays all EC's for all emails in the full conversation related to that EC. Finally, if an attachment is included in the email, a notation for that too is displayed, as seen with the “Attached document” words in EC 1300. It is understood that the notation may be a symbol or combination of words and a symbol, and may be hyperlinked or in a hyperlinked box, such that clicking on those words, symbol or box will pull up the attached document in its native or other format.

Continuing to scroll down (or up) VET 1000 brings FIG. 12B in view, showing 3 more EC's 1400, 1500 and 1600 on the exemplary timeline. As seen here, EC's 1400 and 1500 have been moved to a folder named (or labeled) “Mortgage App” while EC 1600 is in the INBOX (1602).

While with this all-email use case, all emails in a user's email account are represented as individual EC's, thereby presenting the prospect of the inventive system generating a timeline with thousands or tens of thousands of EC's or even more, in practice, one would not be expected to need to scroll through that many EC's to find what she is looking for. First, it is contemplated that users will most often be interested in reviewing relatively recent emails and not very old emails. Thus, presenting the information in reverse chronological order will work well. Second, the invention also contemplates integrating filtering capability with the timeline. Thus, before accessing the “Timeline-All” VET, a user of the present invention with timeline filtering will be able to limit the timeline to generate EC's any of myriad conditions, such as a date range, a topic, only emails with attachments, etc., or limit the all-email timeline to a combination of filtering criteria, as is well understood in the art.

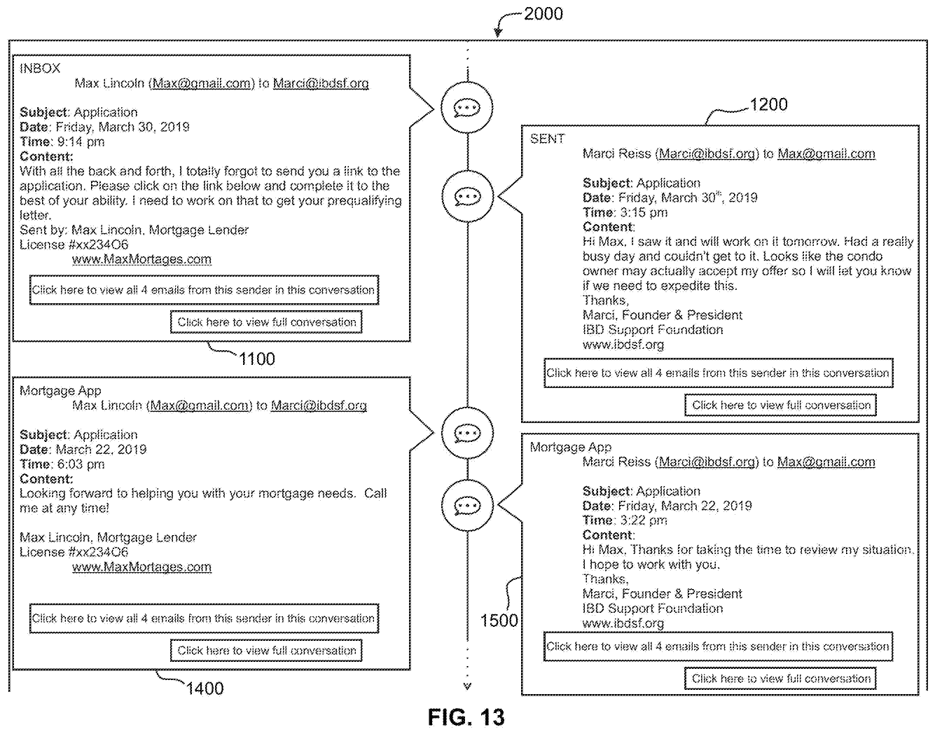

Turning now to FIG. 13, shown is an exemplary VET 2000 that is generated when the user clicks on any of the hyperlinked “Click to view full conversation” boxes in EC's 1100, 1200 1400 or 1500 shown in FIGS. 12B and 12B. As seen, because there are 4 emails in this conversation or email chain or thread, 4 EC cards are generated, giving the user a clean look at the all email traffic in the thread. It will be appreciated that this timeline-in-a-timeline, or “nested VET” capability will have great utility for users with many emails and many conversation threads.

In one embodiment, the present invention is provided as a standalone email client application. In another, the invention is provided in as application that integrates with existing email programs via API's. For example, the present invention can be programmed to query a user's Gmail account by accessing the Gmail API, thereby presenting the novel timelines of the present invention in a native app or as a web-based solution.

The email GUI of the present invention thus has great utility and value in a number of embodiments. Specific to email retrieval systems, the inventive system provides for each easy input of, retrieval of and access to any email stored in an email system. The system allows for an efficient scrolling through of email information with a unique visual display architecture that displays only relevant but sufficient information and content for each email, dramatically reducing the time and effort needed to find emails of interest, thereby significantly improving work efficiency and significantly retrieval time and frustration, and reducing errors and omissions in retrieving record entries.

While embodiments of the invention have been illustrated and described, it is not intended that these embodiments illustrate and describe all possible forms of the invention. Various changes, modifications, and alterations in the teachings of the present invention may be contemplated by those skilled in the art without departing from the intended spirit and scope thereof. It is intended that the present invention encompass such changes and modifications.

What is claimed is:

1. A computer-implemented method for presenting electronic health record (EHR) information for a patient having a Patient Record stored in an EHR data system, the method comprising:
displaying on a screen Patient Encounter Cards (PECs) for the patient, each PEC corresponding to a Patient Encounter Information Entry (PEIE) related to a Patient Encounter (PE) with the patient that was electronically input into the patient's EHR;
wherein each PEC includes PE content extracted from its corresponding PEIE comprising at least (i) key outcome information, (ii) a date for the PE, and (iii) a card title; and
wherein the PECs are presented on the screen in a chronologically ordered timeline.

2. The method of claim 1, wherein at least one PEC contains at least one link or hyperlink to its corresponding PEIE such that upon activation of the link or hyperlink causes the PEIE to be displayed.

3. The method of claim 1, wherein each PEIE comprises a record of any of Progress Notes, Lab Results, Patient Procedure Events, Survey Results, and any combination thereof.

4. The method of claim 3, wherein each PEC on the timeline comprises any of a Progress Note Card, Lab Result Card, Patient Procedure Event Card and Survey Summary Card, each such card corresponding to a respective Progress Note, Lab Result, Patient Procedure Event, or Survey Result.

5. The method of claim 1, wherein the timeline is displayed as a vertical timeline having nodes representing Patient Encounters (PEs), and wherein each node is visually connected to a PEC.

6. The method of claim 5, wherein each node contains visual information indicative of the type of PE represented by its connected PEC.

7. The method of claim 1, wherein the timeline is dynamically created based on filterable criteria selected by the user.

8. The method of claim 7, wherein the filterable criteria include the PE type.

9. The method of claim 1, wherein the PECs are ordered in a forward chronologically-ordered timeline.

10. The method of claim 1, wherein the PECs are ordered in a reverse chronologically-ordered timeline.

11. An apparatus comprising:
a processor; and
a memory that stores computer readable code that, when executed by the processor, causes the processor to: display a first interface, the first interface comprising a plurality of patient encounter cards (PECs) for a given patient, each PEC including content extracted from a patient encounter information entry (PEIE) recorded in a patient record (PR) for the patient of an electronic health record (EHR) system;
wherein the plurality of PECs is ordered chronologically on a timeline.

12. The apparatus of claim 11, wherein the timeline is vertical.

13. The apparatus of claim 12, where the timeline is vertically scrollable for displaying PECs on the timeline.

14. The apparatus of claim 11, wherein at least a portion of each of at least some of the plurality of PECs is selectable such that selection of the portion of one of the selectable PECs causes a second interface to be displayed comprising information from the PEIE corresponding to the patient encounter associated with selected PEC.

15. A computer-implemented method for presenting electronic record information (ERI) for an entity having an Entity Record (ER) stored in an ER data system, the method comprising:
displaying on a screen Entity Encounter Cards (EECs) for the entity, each EEC corresponding to an Entity Encounter Information Entry (EEIE) related to an Entity Encounter (EE) with the entity that was electronically input into the entity's ER;
wherein each EEC includes EE content extracted from its corresponding EEIE comprising at least (i) key outcome information, (ii) a date for the EE, and (iii) a card title; and
wherein the displayed EECs are presented on the screen in a chronologically ordered timeline.

16. The method of claim 15, wherein at least one EEC contains at least one link or hyperlink to its corresponding EEIE such that upon activation of the link or hyperlink causes the EEIE to be displayed.

17. The method of claim 15, wherein the timeline is displayed as a vertical timeline having nodes representing Entity Encounters, and wherein each node is visually connected to an EEC.

18. The method of claim 17, wherein each node contains visual information indicative of the type of EE represented by its connected EEC.

19. The method of claim 15, wherein the timeline is dynamically created based on filterable criteria selected by the user.

20. The method of claim 19, wherein the filterable criteria include the EE type.

21. The method of claim 15, wherein the EECs are ordered in a forward chronologically ordered timeline.

22. The method of claim 15, wherein the EECs are ordered in a reverse chronologically-ordered timeline.

23. An apparatus comprising:
a processor; and
a memory storing computer readable code that, when executed by the processor, causes the processor to: display a first interface, the first interface comprising a plurality of Entity Encounter Cards (EECs) for a given entity, each EEC including content extracted from an entity encounter information entry (EEIE) recorded in an entity record (ER) for the entity of an electronic record data system;
wherein the plurality of EECs displayed on the interface are ordered chronologically on a timeline.

24. The apparatus of claim 23, wherein the displayed timeline is vertically oriented.

25. The apparatus of claim 24, where the timeline scrollable on the interface for continuously displaying EECs on the timeline.

26. The apparatus of claim 25, wherein at least a portion of each of at least some of the plurality of EECs is selectable as a link or hyperlink such that selection of the portion of one of the selectable EEC's causes a second interface to be displayed comprising information from the EEIE corresponding to the specific encounter associated with the selected EEC.

27. A computer-implemented method for presenting email information from emails associated with a first email user account of an email system, the method comprising:
displaying on a screen Email Cards (ECs), each EC corresponding to an email stored for the first user account;
wherein each EC includes email content extracted from its corresponding email comprising at least (i) a date, a timestamp or both identifying when email was sent or received, (ii) a card title, and (iii) selected content from the body of the email;
wherein the ECs are displayed on the screen in a chronologically ordered timeline.

28. The method of claim 27, wherein the timeline is displayed as a vertical and scrollable timeline and each EC is visually connected to the timeline.

29. The method of claim 28, wherein the ECs displayed on the timeline correspond to all emails associated with the user's email account.

30. The method of claim 28, wherein the ECs displayed on the timeline correspond to emails stored in, or associated with, a selected email folder or email label.

31. The method of claim 30, wherein the selected folder or label is any of the user's inbox, a sent folder or a user-created folder.

32. The method of claim 27, wherein at least one EC further includes a first link area containing a link or hyperlink to its corresponding email such that upon activation causes the native email to be displayed.

33. The method of claim 27, wherein at least one EC contains information identifying the number of emails associated with the email represented by the EC.

34. The method of claim 33, wherein the association comprises an email conversation between or among the first user and one or more additional email users in email communication with the first user.

35. The method of claim 27, wherein at least one displayed EC further includes an area having a link or hyperlink that when activated causes a new display of a timeline of EC's associated with all emails in the conversation to be displayed.

36. The method of claim 27, wherein the timeline is dynamically created based on filterable criteria for the emails selected by the user.

37. The method of claim 36, wherein the filterable criteria include any of a “date range,” “sent to” or whether or not there are attachments to the emails.

38. The method of claim 27, wherein the ECs are displayed on a forward chronologically ordered timeline.

39. The method of claim 27, wherein the ECs are displayed on a reverse chronologically ordered timeline.

40. The method of claim 27, further including the step, before the displaying step, of receiving a selection from a user of the email system indicating a request for said timeline of ECs to be displayed.

41. The method of claim 40, wherein the selection from the user is made from an add-on to an existing email system.

42. An apparatus, comprising:
a processor; and
a memory that stores computer readable code that, when executed by the processor, causes the processor to: display a first graphical user interface on a screen, the first interface comprising a plurality of email cards (ECs) corresponding to emails stored for a first user having an email user account of an email system, each EC including content extracted from its corresponding email; wherein the displayed plurality of ECs are chronologically ordered.

43. The apparatus of claim 42, wherein the displayed plurality of ECs are presented on a vertical timeline.

44. The apparatus of claim 43, where the timeline is vertically scrollable on the screen for continuously displaying ECs on the timeline.

45. The apparatus of claim 42, wherein at least a portion of each of at least some of the plurality of ECs is selectable such that selection of the portion of one of the selectable ECs causes a second interface to be displayed comprising information from the email corresponding to the selected EC.
